SEQUENCE LISTING (13 sequences)

SEQ ID NO: 1 - protein, 535 aa, Homo sapiens
MGIVEPGCGDMLTGTEPMPGSDEGRAPGADPQHRYFYPEPGAQDADERRGGGSLGSPYPGGALVPAPPSRFLGAYAYPPRPQAAGFPGAGESFPPPADAEGYQPGEGYAAPDPRAGLYPGPREDYALPAGLEVSGKLRVALNNHLLWSKFNQHQTEMIITKQGRRMFPFLSFTVAGLEPTSHYRMFVDVVLVDQHHWRYQSGKWVQCGKAEGSMPGNRLYVHPDSPNTGAHWMRQEVSFGKLKLTNNKGASNNVTQMIVLQSLHKYQPRLHIVEVNDGEPEAACNASNTHIFTFQETQFIAVTAYQNAEITQLKIDNNPFAKGFRENFESMYTSVDTSIPSPPGPNCQFLGGDHYSPLLPNQYPVPSRFYPDLPGQAKDVVPQAYWLGAPRDHSYEAEFRAVSMKPAFLPSAPGPTMSYYRGQEVLAPGAGWPVAPQYPPKMGPASWFRPMRTLPMEPGPGGSEGRGPEDQGPPLVWTEIAPIRPESSDSGLGEGDSKRRRVSPYPSSGDSSSPAGAPSPFDKEAEGQFYNYFPN

SEQ ID NO: 2 - protein, 686 aa, Homo sapiens
MQLGEQLLVSSVNLPGAHFYPLESARGGSGGSAGHLPSAAPSPQKLDLDKASKKFSGSLSCEAVSGEPAAASAGAPAAMLSDTDAGDAFASAAAVAKPGPPDGRKGSPCGEEELPSAAAAAAAAAAAAAATARYSMDSLSSERYYLQSPGPQGSELAAPCSLFPYQAAAGAPHGPVYPAPNGARYPYGSMLPPGGFPAAVCPPGRAQFGPGAGAGSGAGGSSGGGGGPGTYQYSQGAPLYGPYPGAAAAGSCGGLGGLGVPGSGFRAHVYLCNRPLWLKFHRHQTEMIITKQGRRMFPFLSFNINGLNPTAHYNVFVEVVLADPNHWRFQGGKWVTCGKADNNMQGNKMYVHPESPNTGSHWMRQEISFGKLKLTNNKGANNNNTQMIVLQSLHKYQPRLHIVEVTEDGVEDLNEPSKTQTFTFSETQFIAVTAYQNTDITQLKIDHNPFAKGFRDNYDSSHQIVPGGRYGVQSFFPEPFVNTLPQARYYNGERTVPQTNGLLSPQQSEEVANPPQRWLVTPVQQPGTNKLDISSYESEYTSSTLLPYGIKSLPLQTSHALGYYPDPTFPAMAGWGGRGSYQRKMAAGLPWTSRTSPTVFSEDQLSKEKVKEEIGSSWIETPPSIKSLDSNDSGVYTSACKRRRLSPSNSSNENSPSIKCEDINAEEYSKDTSKGMGGYYAFYTTP

SEQ ID NO: 3 - protein, 498 aa, Homo sapiens
MARRPRHSIYSSDEDDEDFEMCDHDYDGLLPKSGKRHLGKTRWTREEDEKLKKLVEQNGTDDWKVIANYLPNRTDVQCQHRWQKVLNPELIKGPWTKEEDQRVIELVQKYGPKRWSVIAKHLKGRIGKQCRERWHNHLNPEVKKTSWTEEEDRIIYQAHKRLGNRWAEIAKLLPGRTDNAIKNHWNSTMRRKVEQEGYLQMALEDRCSPQSAPSPITLQMQHLHHQQQQQQQQQQQMQHLHQLQQLQQLHQQQLAAGVFHHPAMAFDAAAAAAAAAAAAAAHAHAAALQQRLSGSGSPASCSTPASSTPLTIKEEESDSVIGDMSFHNQTHTTNEEEEAEEDDDIDVDVDDTSAGGRLPPPAHQQQSTAKPSLAFSISNILSDRFGDVQKPGKSIENQASIFRPFEANRSQTATPSAFTRVDLLEFSRQQQAAAAAATAAMMLERANFLNCFNPAAYPRIHEEIVQSRLRRSAANAVIPPPMSSKMSDANPEKSALGS

SEQ ID NO: 4 - protein, 670 aa, Homo sapiens
MGIVEPGCGDMLTGTEPMPGSDEGRAPGADPQHRYFYPEPGAQDADERRGGGSLGSPYPGGALVPAPPSRFLGAYAYPPRPQAAGFPGAGESFPPPADAEGYQPGEGYAAPDPRAGLYPGPREDYALPAGLEVSGKLRVALNNHLLWSKFNQHQTEMIITKQGRRMFPFLSFTVAGLEPTSHYRMFVDVVLVDQHHWRYQSGKWVQCGKAEGSMPGNRLYVHPDSPNTGAHWMRQEVSFGKLKLTNNKGASNNVTQMIVLQSLHKYQPRLHIVEVNDGEPEAACNASNTHIFTFQETQFIAVTAYQNAEITQLKIDNNPFAKGFRENFESMYTSVDTSIPSPPGPNCQFLGGDHYSPLLPNQYPVPSRFYPDMALEDRCSPQSAPSPITLQMQHLHHQQQQQQQQQQQMQHLHQLQQLQQLHQQQLAAGVFHHPAMAFDAAAAAAAAAAAAAAHAHAAALQQRLSGSGSPASCSTPASSTPLTIKEEESDSVIGDMSFHNQTHTTNEEEEAEEDDDIDVDVDDTSAGGRLPPPAHQQQSTAKPSLAFSISNILSDRFGDVQKPGKSIENQASIFRPFEANRSQTATPSAFTRVDLLEFSRQQQAAAAAATAAMMLERANFLNCFNPAAYPRIHEEIVQSRLRRSAANAVIPPPMSSKMSDANPEKSALGS

SEQ ID NO: 5 - protein, 799 aa, Homo sapiens
MQLGEQLLVSSVNLPGAHFYPLESARGGSGGSAGHLPSAAPSPQKLDLDKASKKFSGSLSCEAVSGEPAAASAGAPAAMLSDTDAGDAFASAAAVAKPGPPDGRKGSPCGEEELPSAAAAAAAAAAAAAATARYSMDSLSSERYYLQSPGPQGSELAAPCSLFPYQAAAGAPHGPVYPAPNGARYPYGSMLPPGGFPAAVCPPGRAQFGPGAGAGSGAGGSSGGGGGPGTYQYSQGAPLYGPYPGAAAAGSCGGLGGLGVPGSGFRAHVYLCNRPLWLKFHRHQTEMIITKQGRRMFPFLSFNINGLNPTAHYNVFVEVVLADPNHWRFQGGKWVTCGKADNNMQGNKMYVHPESPNTGSHWMRQEISFGKLKLTNNKGANNNNTQMIVLQSLHKYQPRLHIVEVTEDGVEDLNEPSKTQTFTFSETQFIAVTAYQNTDITQLKIDHNPFAKGFRDNYDSSHQIVPGGRYGVQSFFPEPFVNTLPQARYYNGERTVPQTNGMALEDRCSPQSAPSPITLQMQHLHHQQQQQQQQQQQMQHLHQLQQLQQLHQQQLAAGVFHHPAMAFDAAAAAAAAAAAAAAHAHAAALQQRLSGSGSPASCSTPASSTPLTIKEEESDSVIGDMSFHNQTHTTNEEEEAEEDDDIDVDVDDTSAGGRLPPPAHQQQSTAKPSLAFSISNILSDRFGDVQKPGKSIENQASIFRPFEANRSQTATPSAFTRVDLLEFSRQQQAAAAAATAAMMLERANFLNCFNPAAYPRIHEEIVQSRLRRSAANAVIPPPMSSKMSDANPEKSALGS

SEQ ID NO: 6 - protein, 498 aa, Homo sapiens; identical to SEQ ID NO: 3

SEQ ID NO: 7 - DNA, 331 bp, Artificial Sequence
  Synthetic Construct
agatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccaccatgg

SEQ ID NO: 8 - DNA, 2002 bp, Artificial Sequence
  Synthetic Construct
agatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccaatgggcattgtggaaccgggctgcggcgatatgctgaccggcaccgaaccgatgccgggcagcgatgaaggccgcgcgccgggcgcggatccgcagcatcgctatttttatccggaaccgggcgcgcaggatgcggatgaacgccgcggcggcggcagcctgggcagcccgtatccgggcggcgcgctggtgccggcgccgccgagccgctttctgggcgcgtatgcgtatccgccgcgcccgcaggcggcgggctttccgggcgcgggcgaaagctttccgccgccggcggatgcggaaggctatcagccgggcgaaggctatgcggcgccggatccgcgcgcgggcctgtatccgggcccgcgcgaagattatgcgctgccggcgggcctggaagtgagcggcaaactgcgcgtggcgctgaacaaccatctgctgtggagcaaatttaaccagcatcagaccgaaatgattattaccaaacagggccgccgcatgtttccgtttctgagctttaccgtggcgggcctggaaccgaccagccattatcgcatgtttgtggatgtggtgctggtggatcagcatcattggcgctatcagagcggcaaatgggtgcagtgcggcaaagcggaaggcagcatgccgggcaaccgcctgtatgtgcatccggatagcccgaacaccggcgcgcattggatgcgccaggaagtgagctttggcaaactgaaactgaccaacaacaaaggcgcgagcaacaacgtgacccagatgattgtgctgcagagcctgcataaatatcagccgcgcctgcatattgtggaagtgaacgatggcgaaccggaagcggcgtgcaacgcgagcaacacccatatttttacctttcaggaaacccagtttattgcggtgaccgcgtatcagaacgcggaaattacccagctgaaaattgataacaacccgtttgcgaaaggctttcgcgaaaactttgaaagcatgtataccagcgtggataccagcattccgagcccgccgggcccgaactgccagtttctgggcggcgatcattatagcccgctgctgccgaaccagtatccggtgccgagccgcttttatccggatctgccgggccaggcgaaagatgtggtgccgcaggcgtattggctgggcgcgccgcgcgatcatagctatgaagcggaatttcgcgcggtgagcatgaaaccggcgtttctgccgagcgcgccgggcccgaccatgagctattatcgcggccaggaagtgctggcgccgggcgcgggctggccggtggcgccgcagtatccgccgaaaatgggcccggcgagctggtttcgcccgatgcgcaccctgccgatggaaccgggcccgggcggcagcgaaggccgcggcccggaagatcagggcccgccgctggtgtggaccgaaattgcgccgattcgcccggaaagcagcgatagcggcctgggcgaaggcgatagcaaacgccgccgcgtgagcccgtatccgagcagcggcgatagcagcagcccggcgggcgcgccgagcccgtttgataaagaagcggaaggccagttttataactattttccgaacggcagcggcgcgaccaactttagcctgctgaaacaggcgggcgatgtggaagaaaacccgggcccgccatgg

SEQ ID NO: 9 - DNA, 2407 bp, Artificial Sequence
  Synthetic Construct
agatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccaatgggcattgtggaaccgggctgcggcgatatgctgaccggcaccgaaccgatgccgggcagcgatgaaggccgcgcgccgggcgcggatccgcagcatcgctatttttatccggaaccgggcgcgcaggatgcggatgaacgccgcggcggcggcagcctgggcagcccgtatccgggcggcgcgctggtgccggcgccgccgagccgctttctgggcgcgtatgcgtatccgccgcgcccgcaggcggcgggctttccgggcgcgggcgaaagctttccgccgccggcggatgcggaaggctatcagccgggcgaaggctatgcggcgccggatccgcgcgcgggcctgtatccgggcccgcgcgaagattatgcgctgccggcgggcctggaagtgagcggcaaactgcgcgtggcgctgaacaaccatctgctgtggagcaaatttaaccagcatcagaccgaaatgattattaccaaacagggccgccgcatgtttccgtttctgagctttaccgtggcgggcctggaaccgaccagccattatcgcatgtttgtggatgtggtgctggtggatcagcatcattggcgctatcagagcggcaaatgggtgcagtgcggcaaagcggaaggcagcatgccgggcaaccgcctgtatgtgcatccggatagcccgaacaccggcgcgcattggatgcgccaggaagtgagctttggcaaactgaaactgaccaacaacaaaggcgcgagcaacaacgtgacccagatgattgtgctgcagagcctgcataaatatcagccgcgcctgcatattgtggaagtgaacgatggcgaaccggaagcggcgtgcaacgcgagcaacacccatatttttacctttcaggaaacccagtttattgcggtgaccgcgtatcagaacgcggaaattacccagctgaaaattgataacaacccgtttgcgaaaggctttcgcgaaaactttgaaagcatgtataccagcgtggataccagcattccgagcccgccgggcccgaactgccagtttctgggcggcgatcattatagcccgctgctgccgaaccagtatccggtgccgagccgcttttatccggatatggcgctggaagatcgctgcagcccgcagagcgcgccgagcccgattaccctgcagatgcagcatctgcatcatcagcagcagcagcagcagcagcagcagcagcagatgcagcatctgcatcagctgcagcagctgcagcagctgcatcagcagcagctggcggcgggcgtgtttcatcatccggcgatggcgtttgatgcggcggcggcggcggcggcggcggcggcggcggcggcggcgcatgcgcatgcggcggcgctgcagcagcgcctgagcggcagcggcagcccggcgagctgcagcaccccggcgagcagcaccccgctgaccattaaagaagaagaaagcgatagcgtgattggcgatatgagctttcataaccagacccataccaccaacgaagaagaagaagcggaagaagatgatgatattgatgtggatgtggatgataccagcgcgggcggccgcctgccgccgccggcgcatcagcagcagagcaccgcgaaaccgagcctggcgtttagcattagcaacattctgagcgatcgctttggcgatgtgcagaaaccgggcaaaagcattgaaaaccaggcgagcatttttcgcccgtttgaagcgaaccgcagccagaccgcgaccccgagcgcgtttacccgcgtggatctgctggaatttagccgccagcagcaggcggcggcggcggcggcgaccgcggcgatgatgctggaacgcgcgaactttctgaactgctttaacccggcggcgtatccgcgcattcatgaagaaattgtgcagagccgcctgcgccgcagcgcggcgaacgcggtgattccgccgccgatgagcagcaaaatgagcgatgcgaacccggaaaaaagcgcgctgggcagcggcagcggcgcgaccaactttagcctgctgaaacaggcgggcgatgtggaagaaaacccgggcccgccatgg

SEQ ID NO: 10 - DNA, 2455 bp, Artificial Sequence
  Synthetic Construct
agatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccaatgcagctgggcgaacagctgctggtgagcagcgtgaacctgccgggcgcgcatttttatccgctggaaagcgcgcgcggcggcagcggcggcagcgcgggccatctgccgagcgcggcgccgagcccgcagaaactggatctggataaagcgagcaaaaaatttagcggcagcctgagctgcgaagcggtgagcggcgaaccggcggcggcgagcgcgggcgcgccggcggcgatgctgagcgataccgatgcgggcgatgcgtttgcgagcgcggcggcggtggcgaaaccgggcccgccggatggccgcaaaggcagcccgtgcggcgaagaagaactgccgagcgcggcggcggcggcggcggcggcggcggcggcggcggcggcgaccgcgcgctatagcatggatagcctgagcagcgaacgctattatctgcagagcccgggcccgcagggcagcgaactggcggcgccgtgcagcctgtttccgtatcaggcggcggcgggcgcgccgcatggcccggtgtatccggcgccgaacggcgcgcgctatccgtatggcagcatgctgccgccgggcggctttccggcggcggtgtgcccgccgggccgcgcgcagtttggcccgggcgcgggcgcgggcagcggcgcgggcggcagcagcggcggcggcggcggcccgggcacctatcagtatagccagggcgcgccgctgtatggcccgtatccgggcgcggcggcggcgggcagctgcggcggcctgggcggcctgggcgtgccgggcagcggctttcgcgcgcatgtgtatctgtgcaaccgcccgctgtggctgaaatttcatcgccatcagaccgaaatgattattaccaaacagggccgccgcatgtttccgtttctgagctttaacattaacggcctgaacccgaccgcgcattataacgtgtttgtggaagtggtgctggcggatccgaaccattggcgctttcagggcggcaaatgggtgacctgcggcaaagcggataacaacatgcagggcaacaaaatgtatgtgcatccggaaagcccgaacaccggcagccattggatgcgccaggaaattagctttggcaaactgaaactgaccaacaacaaaggcgcgaacaacaacaacacccagatgattgtgctgcagagcctgcataaatatcagccgcgcctgcatattgtggaagtgaccgaagatggcgtggaagatctgaacgaaccgagcaaaacccagacctttacctttagcgaaacccagtttattgcggtgaccgcgtatcagaacaccgatattacccagctgaaaattgatcataacccgtttgcgaaaggctttcgcgataactatgatagcagccatcagattgtgccgggcggccgctatggcgtgcagagcttttttccggaaccgtttgtgaacaccctgccgcaggcgcgctattataacggcgaacgcaccgtgccgcagaccaacggcctgctgagcccgcagcagagcgaagaagtggcgaacccgccgcagcgctggctggtgaccccggtgcagcagccgggcaccaacaaactggatattagcagctatgaaagcgaatataccagcagcaccctgctgccgtatggcattaaaagcctgccgctgcagaccagccatgcgctgggctattatccggatccgacctttccggcgatggcgggctggggcggccgcggcagctatcagcgcaaaatggcggcgggcctgccgtggaccagccgcaccagcccgaccgtgtttagcgaagatcagctgagcaaagaaaaagtgaaagaagaaattggcagcagctggattgaaaccccgccgagcattaaaagcctggatagcaacgatagcggcgtgtataccagcgcgtgcaaacgccgccgcctgagcccgagcaacagcagcaacgaaaacagcccgagcattaaatgcgaagatattaacgcggaagaatatagcaaagataccagcaaaggcatgggcggctattatgcgttttataccaccccgggcagcggcgcgaccaactttagcctgctgaaacaggcgggcgatgtggaagaaaacccgggcccgccatgg

SEQ ID NO: 11 - DNA, 2794 bp, Artificial Sequence
  Synthetic Construct
agatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccaatgcagctgggcgaacagctgctggtgagcagcgtgaacctgccgggcgcgcatttttatccgctggaaagcgcgcgcggcggcagcggcggcagcgcgggccatctgccgagcgcggcgccgagcccgcagaaactggatctggataaagcgagcaaaaaatttagcggcagcctgagctgcgaagcggtgagcggcgaaccggcggcggcgagcgcgggcgcgccggcggcgatgctgagcgataccgatgcgggcgatgcgtttgcgagcgcggcggcggtggcgaaaccgggcccgccggatggccgcaaaggcagcccgtgcggcgaagaagaactgccgagcgcggcggcggcggcggcggcggcggcggcggcggcggcggcgaccgcgcgctatagcatggatagcctgagcagcgaacgctattatctgcagagcccgggcccgcagggcagcgaactggcggcgccgtgcagcctgtttccgtatcaggcggcggcgggcgcgccgcatggcccggtgtatccggcgccgaacggcgcgcgctatccgtatggcagcatgctgccgccgggcggctttccggcggcggtgtgcccgccgggccgcgcgcagtttggcccgggcgcgggcgcgggcagcggcgcgggcggcagcagcggcggcggcggcggcccgggcacctatcagtatagccagggcgcgccgctgtatggcccgtatccgggcgcggcggcggcgggcagctgcggcggcctgggcggcctgggcgtgccgggcagcggctttcgcgcgcatgtgtatctgtgcaaccgcccgctgtggctgaaatttcatcgccatcagaccgaaatgattattaccaaacagggccgccgcatgtttccgtttctgagctttaacattaacggcctgaacccgaccgcgcattataacgtgtttgtggaagtggtgctggcggatccgaaccattggcgctttcagggcggcaaatgggtgacctgcggcaaagcggataacaacatgcagggcaacaaaatgtatgtgcatccggaaagcccgaacaccggcagccattggatgcgccaggaaattagctttggcaaactgaaactgaccaacaacaaaggcgcgaacaacaacaacacccagatgattgtgctgcagagcctgcataaatatcagccgcgcctgcatattgtggaagtgaccgaagatggcgtggaagatctgaacgaaccgagcaaaacccagacctttacctttagcgaaacccagtttattgcggtgaccgcgtatcagaacaccgatattacccagctgaaaattgatcataacccgtttgcgaaaggctttcgcgataactatgatagcagccatcagattgtgccgggcggccgctatggcgtgcagagcttttttccggaaccgtttgtgaacaccctgccgcaggcgcgctattataacggcgaacgcaccgtgccgcagaccaacggcatggcgctggaagatcgctgcagcccgcagagcgcgccgagcccgattaccctgcagatgcagcatctgcatcatcagcagcagcagcagcagcagcagcagcagcagatgcagcatctgcatcagctgcagcagctgcagcagctgcatcagcagcagctggcggcgggcgtgtttcatcatccggcgatggcgtttgatgcggcggcggcggcggcggcggcggcggcggcggcggcggcgcatgcgcatgcggcggcgctgcagcagcgcctgagcggcagcggcagcccggcgagctgcagcaccccggcgagcagcaccccgctgaccattaaagaagaagaaagcgatagcgtgattggcgatatgagctttcataaccagacccataccaccaacgaagaagaagaagcggaagaagatgatgatattgatgtggatgtggatgataccagcgcgggcggccgcctgccgccgccggcgcatcagcagcagagcaccgcgaaaccgagcctggcgtttagcattagcaacattctgagcgatcgctttggcgatgtgcagaaaccgggcaaaagcattgaaaaccaggcgagcatttttcgcccgtttgaagcgaaccgcagccagaccgcgaccccgagcgcgtttacccgcgtggatctgctggaatttagccgccagcagcaggcggcggcggcggcggcgaccgcggcgatgatgctggaacgcgcgaactttctgaactgctttaacccggcggcgtatccgcgcattcatgaagaaattgtgcagagccgcctgcgccgcagcgcggcgaacgcggtgattccgccgccgatgagcagcaaaatgagcgatgcgaacccggaaaaaagcgcgctgggcagcggcagcggcgcgaccaactttagcctgctgaaacaggcgggcgatgtggaagaaaacccgggcccgccatgg

SEQ ID NO: 12 - DNA, 2317 bp, Artificial Sequence
  Synthetic Construct
agatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccaatggcgcgccgcccgcgccatagcatttatagcagcgatgaagatgatgaagattttgaaatgtgcgatcatgattatgatggcctgctgccgaaaagcggcaaacgccatctgggcaaaacccgctggacccgcgaagaagatgaaaaactgaaaaaactggtggaacagaacggcaccgatgattggaaagtgattgcgaactatctgccgaaccgcaccgatgtgcagtgccagcatcgctggcagaaagtgctgaacccggaactgattaaaggcccgtggaccaaagaagaagatcagcgcgtgattgaactggtgcagaaatatggcccgaaacgctggagcgtgattgcgaaacatctgaaaggccgcattggcaaacagtgccgcgaacgctggcataaccatctgaacccggaagtgaaaaaaaccagctggaccgaagaagaagatcgcattatttatcaggcgcataaacgcctgggcaaccgctgggcggaaattgcgaaactgctgccgggccgcaccgataacgcgattaaaaaccattggaacagcaccatgcgccgcaaagtggaacaggaaggctatctgcaggaaagcagcaaagcgagccagccggcggtggcgaccagctttcagaaaaacagccatctgatgggctttgcgcaggcgccgccgaccgcgcagctgccggcgaccggccagccgaccgtgaacaacgattatagctattatcatattagcgaagcgcagaacgtgagcagccatgtgccgtatccggtggcgctgcatgtgaacattgtgaacgtgccgcagccggcggcggcggcgattcagcgccattataacgatgaagatccggaaaaagaaaaacgcattaaagaactggaactgctgctgatgagcaccgaaaacgaactgaaaggccagcaggtgctgccgacccagaaccatacctgcagctatccgggctggcatagcaccaccattgcggatcatacccgcccgcatggcgatagcgcgccggtgagctgcctgggcgaacatcatagcaccccgagcctgccggcggatccgggcagcctgccggaagaaagcgcgagcccggcgcgctgcatgattgtgcatcagggcaccattctggataacgtgaaaaacctgctggaatttgcggaaaccctgcagtttattgatagctttctgaacaccagcagcaaccatgaaaacagcgatctggaaatgccgagcctgaccagcaccccgctgattggccataaactgaccgtgaccaccccgtttcatcgcgatcagaccgtgaaaacccagaaagaaaacaccgtgtttcgcaccccggcgattaaacgcagcattctggaaagcagcccgcgcaccccgaccccgtttaaacatgcgctggcggcgcaggaaattaaatatggcccgctgaaaatgctgccgcagaccccgagccatctggtggaagatctgcaggatgtgattaaacaggaaagcgatgaaagcggcattgtggcggaatttcaggaaaacggcccgccgctgctgaaaaaaattaaacaggaagtggaaagcccgaccgataaaagcggcaactttttttgcagccatcattgggaaggcgatagcctgaacacccagctgtttacccagaccagcccggtggcggatgcgccgaacattctgaccagcagcgtgctgatggcgccggcgagcgaagatgaagataacgtgctgaaagcgtttaccgtgccgaaaaaccgcagcctggcgagcccgctgcagccgtgcagcagcacctgggaaccggcgagctgcggcaaaatggaagaacagatgaccagcagcagccaggcgcgcaaatatgtgaacgcgtttagcgcgcgcaccctggtgatgggcagcggcgcgaccaactttagcctgctgaaacaggcgggcgatgtggaagaaaacccgggcccgccatgg

SEQ ID NO: 13 - DNA, 1891 bp, Artificial Sequence
  Synthetic Construct
agatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccaatggcgcgccgcccgcgccatagcatttatagcagcgatgaagatgatgaagattttgaaatgtgcgatcatgattatgatggcctgctgccgaaaagcggcaaacgccatctgggcaaaacccgctggacccgcgaagaagatgaaaaactgaaaaaactggtggaacagaacggcaccgatgattggaaagtgattgcgaactatctgccgaaccgcaccgatgtgcagtgccagcatcgctggcagaaagtgctgaacccggaactgattaaaggcccgtggaccaaagaagaagatcagcgcgtgattgaactggtgcagaaatatggcccgaaacgctggagcgtgattgcgaaacatctgaaaggccgcattggcaaacagtgccgcgaacgctggcataaccatctgaacccggaagtgaaaaaaaccagctggaccgaagaagaagatcgcattatttatcaggcgcataaacgcctgggcaaccgctgggcggaaattgcgaaactgctgccgggccgcaccgataacgcgattaaaaaccattggaacagcaccatgcgccgcaaagtggaacaggaaggctatctgcagatggcgctggaagatcgctgcagcccgcagagcgcgccgagcccgattaccctgcagatgcagcatctgcatcatcagcagcagcagcagcagcagcagcagcagcagatgcagcatctgcatcagctgcagcagctgcagcagctgcatcagcagcagctggcggcgggcgtgtttcatcatccggcgatggcgtttgatgcggcggcggcggcggcggcggcggcggcggcggcggcggcgcatgcgcatgcggcggcgctgcagcagcgcctgagcggcagcggcagcccggcgagctgcagcaccccggcgagcagcaccccgctgaccattaaagaagaagaaagcgatagcgtgattggcgatatgagctttcataaccagacccataccaccaacgaagaagaagaagcggaagaagatgatgatattgatgtggatgtggatgataccagcgcgggcggccgcctgccgccgccggcgcatcagcagcagagcaccgcgaaaccgagcctggcgtttagcattagcaacattctgagcgatcgctttggcgatgtgcagaaaccgggcaaaagcattgaaaaccaggcgagcatttttcgcccgtttgaagcgaaccgcagccagaccgcgaccccgagcgcgtttacccgcgtggatctgctggaatttagccgccagcagcaggcggcggcggcggcggcgaccgcggcgatgatgctggaacgcgcgaactttctgaactgctttaacccggcggcgtatccgcgcattcatgaagaaattgtgcagagccgcctgcgccgcagcgcggcgaacgcggtgattccgccgccgatgagcagcaaaatgagcgatgcgaacccggaaaaaagcgcgctgggcagcggcagcggcgcgaccaactttagcctgctgaaacaggcgggcgatgtggaagaaaacccgggcccgccatgg

(19) United States
(12) Patent Application Publication
(10) Pub. No.: US 2022/0228114 A1
(43) Pub. Date: Jul. 21, 2022

(54) THERAPEUTIC T-CELLS WITH MODIFIED EXPRESSION OF T-BET, EOMES, AND c-MYB TRANSCRIPTION FACTORS

(71) Applicant: H. Lee Moffitt Cancer Center and Research Institute, Inc., Tampa, FL (US)

(72) Inventor: Marco L. Davila, Tampa, FL (US)

(21) Appl. No.: 17/614,936

(22) PCT Filed: May 29, 2020
(86) PCT No.: PCT/US2020/035272

§ 371 Date: Nov. 29, 2021

Related U.S. Application Data
(60) Provisional application No. 62/854,228, filed on May 29, 2019.

(51) Int. Cl. C12N 5/0783; C12N 15/86; C07K 14/47; A61K 35/17; A61K 39/395

(52) U.S. Cl. CPC C12N 5/0636; C12N 15/86; C07K 14/4702; C12N 2510/00; A61K 39/3955; C12N 2740/10043; A61K 35/17

(57) ABSTRACT

Disclosed herein are recombinant immune effector cells genetically modified to regulate expression of one or more transcription factors selected from T-bet, Eomes, and c-Myb. In some embodiments, the cells are modified to express recombinant T-bet, Eomes, c-Myb, or any combination thereof. In some embodiments, the cells are modified to express recombinant dominant negative forms of T-bet, Eomes, c-Myb, or any combination thereof to inhibit the activity of endogenous transcription factors. In some embodiments combinations of these approaches are used to increase expression of one or more of T-bet, Eomes, c-Myb while inhibiting the activity of one or more of T-bet, Eomes, and c-Myb.

### CROSS-REFERENCE TO RELATED APPLICATIONS

This application claims benefit of U.S. Provisional Application No. 62/854,228, filed May 29, 2019, which is hereby incorporated herein by reference in its entirety.

### SEQUENCE LISTING

This application contains a sequence listing filed in electronic form as an ASCII.txt file entitled “320803_2350_Sequence_Listing_ST25” created on May 27, 2020. The content of the sequence listing is incorporated herein in its entirety.

### BACKGROUND

Surgery, radiation therapy, and chemotherapy have been the standard accepted approaches for treatment of cancers including leukemia, solid tumors, and metastases. Immunotherapy (sometimes called biological therapy, biotherapy, or biological response modifier therapy), which uses the body's immune system, either directly or indirectly, to shrink or eradicate cancer has been studied for many years as an adjunct to conventional cancer therapy. It is believed that the human immune system is an untapped resource for cancer therapy and that effective treatment can be developed once the components of the immune system are properly harnessed.

### SUMMARY

Disclosed is an ex vivo method for reversing intrinsic defects of aged immune effector cells, such as terminal differentiation and loss of central memory cells. In some embodiments, the disclosed methods involve genetically modifying the cells with modified T-bet, Eomes, and/or c-Myb expression or activity. This method reverses terminal differentiation, thereby increasing the anti-tumor activity and memory differentiation of the immune effector cells. For example, transduction of aged T cells with Tbet skews the CD4/CD8 ratio towards maintaining CD4 along with an increase in central memory cells and a decrease in effector memory cells profiling a similar phenotype of young Tbet-armed CAR-T cells.

As used herein, aged immune effector cells are cells from a donor that is at least 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 61, 62, 64, 65, 66, 67, 68, 69, or 70 years old. Aged cells have a lower CD4/CD8 ratio and decreased central memory cells when activated, e.g. with CD3/CD28 beads. Therefore, in some embodiments, the donor immune effector cells have a CD4/CD8 ratio less than 0.5, 0.4, 0.3, 0.2, or 0.1 when activated, and the disclosed methods increase the CD4/CD8 ratio to higher than 0.5, 0.6, 0.8, 0.9, 1.0, or higher when activated. In addition, aged cells have elevated terminally differentiated cells. TILs are typically terminally differentiated cells; therefore, in some embodiments, the disclosed methods can also be used to increase anti-cancer activity of TILs.

Therefore, also disclosed is an ex vivo method for enhancing anti-tumor efficacy of immune effector cells, comprising genetically modifying the immune effector cells to regulate expression of one or more transcription factors selected from T-bet, Eomes, and c-Myb, in an amount effective to increase CD4/CD8 ratio by at least 0.1, 0.2, 0.3, 0.4, 0.5, 0.6, 0.7, 0.8, 0.9, 1, 2, 3, 4, 5, 6, 7, 8, 9, 10, 11, 12, 13, 14, or 15 when activated. In some embodiments, the immune effector cells are aged immune effector cells, tumor infiltrating lymphocyte (TIL), or a combination thereof.

The disclosed recombinant immune effector cells can be genetically modified to regulate expression of one or more transcription factors selected from T-bet, Eomes, and c-Myb. In some embodiments, the cells are modified to express recombinant T-bet, Eomes, c-Myb, or any combination thereof. In some embodiments, the cells are modified to express recombinant dominant negative forms of T-bet, Eomes, c-Myb, or any combination thereof to inhibit the activity of endogenous transcription factors. In some embodiments combinations of these approaches are used to increase expression of one or more of T-bet, Eomes, c-Myb while inhibiting the activity of one or more of T-bet, Eomes, and c-Myb.

Therefore, the disclosed aged recombinant immune effector cells can express recombinant T-bet; recombinant Eomes; recombinant c-Myb; recombinant T-bet and recombinant Eomes; recombinant T-bet and recombinant c-Myb; recombinant Eomes and recombinant c-Myb; recombinant T-bet, recombinant Eomes, and recombinant c-Myb; dominant negative T-bet; dominant negative Eomes; dominant negative c-Myb; dominant negative T-bet and dominant negative Eomes; dominant negative T-bet and dominant negative c-Myb; dominant negative Eomes and dominant negative c-Myb; dominant negative T-bet, dominant negative Eomes, and dominant negative c-Myb; recombinant T-bet and dominant negative Eomes; recombinant Eomes and dominant negative T-bet; recombinant T-bet and dominant negative c-Myb; recombinant c-Myb and dominant negative Eomes; recombinant c-Myb and dominant negative Eomes; recombinant Eomes and dominant negative c-Myb; recombinant T-bet, recombinant Eomes, and dominant negative c-Myb; recombinant Eomes, recombinant c-Myb, and dominant negative T-bet; recombinant c-Myb, recombinant T-bet, and dominant negative Eomes; recombinant T-bet, dominant negative Eomes, and dominant negative c-Myb; recombinant Eomes, dominant negative c-Myb, and dominant negative T-bet; or recombinant c-Myb, dominant negative T-bet, and dominant negative Eomes.

Nucleic acid and amino acid sequences for T-bet, Eomes, and c-Myb are known in the art and can be adapted for recombinant expression, including fragments and variants that are biologically active.

In some embodiments, human T-bet has the amino acid sequence

(SEQ ID NO: 1)
MGIVEPGCGDMLTGTEPMPGSDEGRAPGADPQHRYFYPEPGAQDADERRG
GGSLGSPYPGGALVPAPPSRFLGAYAYPPRPQAAGFPGAGESFPPPADAE
GYQPGEGYAAPDPRAGLYPGPREDYALPAGLEVSGKLRVALNNHLLWSKF
NQHQTEMIITKQGRRMFPFLSFTVAGLEPTSHYRMFVDVVLVDQHHWRYQ
SGKWVQCGKAEGSMPGNRLYVHPDSPNTGAHVVMRQEVSFGKLKLTNNKG
ASNNVTQMIVLQSLHKYQPRLHIVEVNDGEPEAACNASNTHIFTFQETQF
IAVTAYQNAEITQLKIDNNPFAKGFRENFESMYTSVDTSIPSPPGPNCQF
LGGDHYSPLLPNQYPVPSRFYPDLPGQAKDVVPQAYWLGAPRDHSYEAEF
RAVSMKPAFLPSAPGPTMSYYRGQEVLAPGAGWPVAPQYPPKMGPASWFR
PMRTLPMEPGPGGSEGRGPEDQGPPLVWTEIAPIRPESSDSGLGEGDSKR
RRVSPYPSSGDSSSPAGAPSPFDKEAEGQFYNYFPN.

In some embodiments, human Eomes has the amino acid sequence

(SEQ ID NO: 2)
MQLGEQLLVSSVNLPGAHFYPLESARGGSGGSAGHLPSAAPSPQKLDLDK
ASKKFSGSLSCEAVSGEPAAASAGAPAAMLSDTDAGDAFASAAAVAKPGP
PDGRKGSPCGEEELPSAAAAAAAAAAAAAATARYSMDSLSSERYYLQSPG
PQGSELAAPCSLFPYQAAAGAPHGPVYPAPNGARYPYGSMLPPGGFPAAV
CPPGRAQFGPGAGAGSGAGGSSGGGGGPGTYQYSQGAPLYGPYPGAAAAG
SCGGLGGLGVPGSGFRAHVYLCNRPLWLKFHRHQTEMIITKQGRRMFPFL
SFNINGLNPTAHYNVFVEVVLADPNHWRFQGGKWVTCGKADNNMQGNKMY
VHPESPNTGSHWMRQEISFGKLKLTNNKGANNNNTQMIVLQSLHKYQPRL
HIVEVTEDGVEDLNEPSKTQTFTFSETQFIAVTAYQNTDITQLKIDHNPF
AKGFRDNYDSSHQIVPGGRYGVQSFFPEPFVNTLPQARYYNGERTVPQTN
GLLSPQQSEEVANPPQRWLVTPVQQPGTNKLDISSYESEYTSSTLLPYGI
KSLPLQTSHALGYYPDPTFPAMAGWGGRGSYQRKMAAGLPWTSRTSPTVF
SEDQLSKEKVKEEIGSSWIETPPSIKSLDSNDSGVYTSACKRRRLSPSNS
SNENSPSIKCEDINAEEYSKDTSKGMGGYYAFYTTP.

In some embodiments, human c-Myb has the amino acid sequence

(SEQ ID NO: 3)
MARRPRHSIYSSDEDDEDFEMCDHDYDGLLPKSGKRHLGKTRWTREEDEK
LKKLVEQNGTDDWKVIANYLPNRTDVQCQHRWQKVLNPELIKGPVVTKEE
DQRVIELVQKYGPKRWSVIAKHLKGRIGKQCRERWHNHLNPEVKKTSWTE
EEDRIIYQAHKRLGNRWAEIAKLLPGRTDNAIKNHWNSTMRRKVEQEGYL
QMALEDRCSPQSAPSPITLQMQHLHHQQQQQQQQQQQMQHLHQLQQLQQL
HQQQLAAGVFHHPAMAFDAAAAAAAAAAAAAAHAHAAALQQRLSGSGSPA
SCSTPASSTPLTIKEEESDSVIGDMSFHNQTHTTNEEEEAEEDDDIDVDV
DDTSAGGRLPPPAHQQQSTAKPSLAFSISNILSDRFGDVQKPGKSIENQA
SIFRPFEANRSQTATPSAFTRVDLLEFSRQQQAAAAAATAAMMLERANFL
NCFNPAAYPRIHEEIVQSRLRRSAANAVIPPPMSSKMSDANPEKSALGS.

Methods for making dominant negative forms of transcription factors are known in the art. For example, in some embodiments, as depicted in FIG. 2, the transactivating domain can be deleted, mutated, or replaced such that the dominant negative construct is inactive but competes with endogenous transcription factors. In some embodiments, the transactivating domain is replaced with a repressor domain, such as the engrailed gene from Drosophila.

In some embodiments, a dominant form of human T-bet has the amino acid sequence

(SEQ ID NO: 4)
MGIVEPGCGDMLTGTEPMPGSDEGRAPGADPQHRYFYPEPGAQDADERRG
GGSLGSPYPGGALVPAPPSRFLGAYAYPPRPQAAGFPGAGESFPPPADAE
GYQPGEGYAAPDPRAGLYPGPREDYALPAGLEVSGKLRVALNNHLLWSKF
NQHQTEMIITKQGRRMFPFLSFTVAGLEPTSHYRMFVDVVLVDQHHWRYQ
SGKWVQCGKAEGSMPGNRLYVHPDSPNTGAHWMRQEVSFGKLKLTNNKGA
SNNVTQMIVLQSLHKYQPRLHIVEVNDGEPEAACNASNTHIFTFQETQFI
AVTAYQNAEITQLKIDNNPFAKGFRENFESMYTSVDTSIPSPPGPNCQFL
GGDHYSPLLPNQYPVPSRFYPDMALEDRCSPQSAPSPITLQMQHLHHQQQ
QQQQQQQQMQHLHQLQQLQQLHQQQLAAGVFHHPAMAFDAAAAAAAAAAA
AAAHAHAAALQQRLSGSGSPASCSTPASSTPLTIKEEESDSVIGDMSFHN
QTHTTNEEEEAEEDDDIDVDVDDTSAGGRLPPPAHQQQSTAKPSLAFSIS
NILSDRFGDVQKPGKSIENQASIFRPFEANRSQTATPSAFTRVDLLEFSR
QQQAAAAAATAAMMLERANFLNCFNPAAYPRIHEEIVQSRLRRSAANAVI
PPPMSSKMSDANPEKSALGS.

In some embodiments, a dominant form of human Eomes has the amino acid sequence

(SEQ ID NO: 5)
MQLGEQLLVSSVNLPGAHFYPLESARGGSGGSAGHLPSAAPSPQKLDLD
KASKKFSGSLSCEAVSGEPAAASAGAPAAMLSDTDAGDAFASAAAVAKP
GPPDGRKGSPCGEEELPSAAAAAAAAAAAAAATARYSMDSLSSERYYLQ
SPGPQGSELAAPCSLFPYQAAAGAPHGPVYPAPNGARYPYGSMLPPGGF
PAAVCPPGRAQFGPGAGAGSGAGGSSGGGGGPGTYQYSQGAPLYGPYPG
AAAAGSCGGLGGLGVPGSGFRAHVYLCNRPLWLKFHRHQTEMIITKQGR
RMFPFLSFNINGLNPTAHYNVFVEVVLADPNHWRFQGGKVVVTCGKADN
NMQGNKMYVHPESPNTGSHWMRQEISFGKLKLTNNKGANNNNTQMIVLQ
SLHKYQPRLHIVEVTEDGVEDLNEPSKTQTFTFSETQFIAVTAYQNTDI
TQLKIDHNPFAKGFRDNYDSSHQIVPGGRYGVQSFFPEPFVNTLPQARY
YNGERTVPQTNGMALEDRCSPQSAPSPITLQMQHLHHQQQQQQQQQQQM
QHLHQLQQLQQLHQQQLAAGVFHHPAMAFDAAAAAAAAAAAAAAHAHAA
ALQQRLSGSGSPASCSTPASSTPLTIKEEESDSVIGDMSFHNQTHTTNE
EEEAEEDDDIDVDVDDTSAGGRLPPPAHQQQSTAKPSLAFSISNILSDR
FGDVQKPGKSIENQASIFRPFEANRSQTATPSAFTRVDLLEFSRQQQAA
AAAATAAMMLERANFLNCFNPAAYPRIHEEIVQSRLRRSAANAVIPPPM
SSKMSDANPEKSALGS.

In some embodiments, a dominant form of human c-Myb has the amino acid sequence

(SEQ ID NO: 6)
MARRPRHSIYSSDEDDEDFEMCDHDYDGLLPKSGKRHLGKTRVVTREEDE
KLKKLVEQNGTDDWKVIANYLPNRTDVQCQHRWQKVLNPELIKGPWTKEE
DQRVIELVQKYGPKRWSVIAKHLKGRIGKQCRERWHNHLNPEVKKTSWTE
EEDRIIYQAHKRLGNRWAEIAKLLPGRTDNAIKNHWNSTMRRKVEQEGYL
QMALEDRCSPQSAPSPITLQMQHLHHQQQQQQQQQQQMQHLHQLQQLQQL
HQQQLAAGVFHHPAMAFDAAAAAAAAAAAAAAHAHAAALQQRLSGSGSPA
SCSTPASSTPLTIKEEESDSVIGDMSFHNQTHTTNEEEEAEEDDDIDVDV
DDTSAGGRLPPPAHQQQSTAKPSLAFSISNILSDRFGDVQKPGKSIENQA
SIFRPFEANRSQTATPSAFTRVDLLEFSRQQQAAAAAATAAMMLERANFL
NCFNPAAYPRIHEEIVQSRLRRSAANAVIPPPMSSKMSDANPEKSALGS.

In some embodiments, the aged immune effector cells also express chimeric antigen receptor (CAR) polypeptides or transgenic TCRs, and can be used with adoptive cell transfer to target and kill cancers.

In some embodiments, the recombinant immune effector cell comprises a vector containing nucleic acid sequences encoding the above transcription factors and/or dominant negative forms of the transcription factors operably linked to expression control sequences. In some embodiments, the vector further contains nucleic acid sequences encoding a CAR operably linked to an expression control sequence. The nucleic acid sequences encoding the above transcription factors and/or dominant negative forms of the transcription factors and the nucleic acid sequences encoding a CAR can in some embodiments be operably linked to the same expression control sequence. For example, the nucleic acid sequences can be separated by a IRES or P2A cleavage site.

In some embodiments, the immune effector cell is selected from the group consisting of an alpha-beta T cells, a gamma-delta T cell, a Natural Killer (NK) cells, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, and a regulatory T cell. In some embodiments, the immune effector cell is a CAR-T cells.

In some embodiments, the cell further comprises a molecular suicide switch system to remove the transferred cell population. For example, the nucleic acid encoding the CAR polypeptide can be part of an expression cassette that also includes an accessory gene. For example, in some embodiments, the accessory gene is a truncated EGFR gene (EGFRt). An EGFRt may be used as a non-immunogenic selection tool (e.g., immunomagnetic selection using biotinylated cetuximab in combination with anti-biotin microbeads for enrichment of T cells that have been lentivirally transduced with EGFRt-containing constructs), tracking marker (e.g., flow cytometric analysis for tracking T cell engraftment), or a suicide gene (e.g., via Cetuximab/Erbitux® mediated antibody dependent cellular cytotoxicity (ADCC) pathways). An example of a truncated EGFR (EGFRt) gene that may be used in accordance with the embodiments described herein is described in International Application No. PCT/US2010/055329, the subject matter of which is hereby incorporated by reference as if fully set forth herein. In other embodiments, the accessory gene is a truncated CD19 gene (CD19t). In other embodiments, the accessory gene is an inducible caspase 9 gene.

Also disclosed is a method of providing an anti-tumor immunity in a subject that involves administering to the subject an effective amount of the disclosed recombinant immune effector cell genetically modified with modified T-bet, Eomes, and/or c-Myb expression or activity.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 shows effect of T-bet expression on m19BBz CAR-T cells.

FIG. 2 illustrates example transcription factors and dominant negative transcription factors for T-bet, Eomes, and c-Myb.

FIGS. 3A to 3D show Tbet-armed CAR exhibited unique functions via maintaining CD4 and memory-like T cells. FIG. 3A shows representation of mouse CD19-targeted CAR constructs: scFV (single-chain variable fragment), CD8L (mouse CD8 leader), IgH (immunoglobylin heavy chains), Gly-Ser (glycine-serine linker), IgL (immunoglobulin light chains), LTR (long terminal repeats), CD8TM (mouse CD8 transmembrane region), CD28 (mouse CD28 co-stimulatory domain), 4-1BB (human 4-1BB co-stimulatory domain), CD3z (CD3 ζ chain), P2A (2A self-cleaving peptides) and Tbet (mouse T-box transcription factor Tbx21). FIG. 3B shows CAR transduction efficiencies (left) and Tbet expression gated on CAR+ cells (right). Data are representative of independent experiments. FIG. 3C shows summarized immune phenotypes in each group. FIG. 3D shows cytokine production. CAR T cells were co-cultured with 3T3 (antigen-) or 3T3-mCD19 (antigen+) at 10:1 E:T ratio for 24 hours. Supernatant were collected and cytokines were measured using Ella. Bar graphs shown as mean±% CV. For FIGS. 3B-3D shows Untrans (untransduced T cells), Young cony. (young B6 derived conventional CAR T cells), Young Tbet-armed (young B6 derived Tbet-armed CAR T cells), Aged cony. (aged B6 derived conventional CAR T cells) and Aged Tbet-armed (aged B6 derived Tbet-armed CAR T cells).

FIGS. 4A and 4B show cytotoxicity comparison of conventional mouse CAR T with Tbet-armed mouse CAR T. Cytotoxicity was monitored by xCELLigence RTCA system as well as FIG. 2D. For FIGS. 4A and 4B show CART cells co-cultured with 3T3-mCD19 at the indicated ratios. Data is representative in triplicate.

FIGS. 5A to 5C show human T cells transduced with Tbet armed CAR skew the CD4/CD8 ratio towards CD4. FIG. 5A is a scheme of the different CAR constructs. A sequence encoding for human Tbet was introduced into a CD19-specific CAR with a 4-1BB as costimulatory domain using the self-cleaving peptide P2A. FIG. 5B shows flow cytometric analysis showing expression of CAR among live CD3+ cells for the different constructs (left). Tbet expression among CAR+ cells (right). Evaluated on unstimulated cells 7 days after initial transduction. Untransduced T (UT) cells were used as control. FIG. 5C shows frequency of CD4+ and CD8+ cells among CD3+ cells and CD3+CAR+ cells.

FIG. 6 shows the cell phenotype is affected by overexpression of Tbet on human T/CAR-T cells with an increase in the memory T cell subset. Frequency of central memory (CM), Naïve (N), effector memory (EM) and terminally differentiated effector memory cells (TEM RA) among total CD3 (top), CD8+(middle) and CD4+(bottom) cells was evaluated on unstimulated cells 7 days after initial transduction.

FIGS. 7A and 7B show Tbet armed CAR-T cells show comparable in vitro cytotoxicity as the conventional counterpart. A real-time cytotoxicity assay (RTCA) using xCELLigence® was performed with the different T/CAR-T cells against immobilized Nalm6 tumor cells at 1:1 and 3:1 effector:target (E:T) ratios.

FIGS. 8A to 8D show cytokine secretion is modified in Tbet armed CAR-T cells. Human CAR-T cells were cultured alone or with Nalm6 tumor cells at a 1:1 ratio for 24 h, supernatants were collected and analyzed for cytokines using ELLA multiplex assay.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The term “amino acid sequence” refers to a list of abbreviations, letters, characters or words representing amino acid residues. The amino acid abbreviations used herein are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

The term “antibody” refers to an immunoglobulin, derivatives thereof which maintain specific binding ability, and proteins having a binding domain which is homologous or largely homologous to an immunoglobulin binding domain. These proteins may be derived from natural sources, or partly or wholly synthetically produced. An antibody may be monoclonal or polyclonal. The antibody may be a member of any immunoglobulin class from any species, including any of the human classes: IgG, IgM, IgA, IgD, and IgE. In exemplary embodiments, antibodies used with the methods and compositions described herein are derivatives of the IgG class. In addition to intact immunoglobulin molecules, also included in the term “antibodies” are fragments or polymers of those immunoglobulin molecules, and human or humanized versions of immunoglobulin molecules that selectively bind the target antigen.

The term “antibody fragment” refers to any derivative of an antibody which is less than full-length. In exemplary embodiments, the antibody fragment retains at least a significant portion of the full-length antibody's specific binding ability. Examples of antibody fragments include, but are not limited to, Fab, Fab′, F(ab′)2, scFv, Fv, dsFv diabody, Fc, and Fd fragments. The antibody fragment may be produced by any means. For instance, the antibody fragment may be enzymatically or chemically produced by fragmentation of an intact antibody, it may be recombinantly produced from a gene encoding the partial antibody sequence, or it may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex. A functional antibody fragment will typically comprise at least about 50 amino acids and more typically will comprise at least about 200 amino acids.

The term “antigen binding site” refers to a region of an antibody that specifically binds an epitope on an antigen.

The term “aptamer” refers to oligonucleic acid or peptide molecules that bind to a specific target molecule. These molecules are generally selected from a random sequence pool. The selected aptamers are capable of adapting unique tertiary structures and recognizing target molecules with high affinity and specificity. A “nucleic acid aptamer” is a DNA or RNA oligonucleic acid that binds to a target molecule via its conformation, and thereby inhibits or suppresses functions of such molecule. A nucleic acid aptamer may be constituted by DNA, RNA, or a combination thereof. A “peptide aptamer” is a combinatorial protein molecule with a variable peptide sequence inserted within a constant scaffold protein. Identification of peptide aptamers is typically performed under stringent yeast dihybrid conditions, which enhances the probability for the selected peptide aptamers to be stably expressed and correctly folded in an intracellular context.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. For example, a carrier can be selected to minimize any degradation of the active ingredient and to minimize any adverse side effects in the subject.

The term “chimeric molecule” refers to a single molecule created by joining two or more molecules that exist separately in their native state. The single, chimeric molecule has the desired functionality of all of its constituent molecules. One type of chimeric molecules is a fusion protein.

The term “engineered antibody” refers to a recombinant molecule that comprises at least an antibody fragment comprising an antigen binding site derived from the variable domain of the heavy chain and/or light chain of an antibody and may optionally comprise the entire or part of the variable and/or constant domains of an antibody from any of the Ig classes (for example IgA, IgD, IgE, IgG, IgM and IgY).

The term “epitope” refers to the region of an antigen to which an antibody binds preferentially and specifically. A monoclonal antibody binds preferentially to a single specific epitope of a molecule that can be molecularly defined. In the present invention, multiple epitopes can be recognized by a multispecific antibody.

The term “fusion protein” refers to a polypeptide formed by the joining of two or more polypeptides through a peptide bond formed between the amino terminus of one polypeptide and the carboxyl terminus of another polypeptide. The fusion protein can be formed by the chemical coupling of the constituent polypeptides or it can be expressed as a single polypeptide from nucleic acid sequence encoding the single contiguous fusion protein. A single chain fusion protein is a fusion protein having a single contiguous polypeptide backbone. Fusion proteins can be prepared using conventional techniques in molecular biology to join the two genes in frame into a single nucleic acid, and then expressing the nucleic acid in an appropriate host cell under conditions in which the fusion protein is produced.

The term “Fab fragment” refers to a fragment of an antibody comprising an antigen-binding site generated by cleavage of the antibody with the enzyme papain, which cuts at the hinge region N-terminally to the inter-H-chain disulfide bond and generates two Fab fragments from one antibody molecule.

The term “F(ab′)2 fragment” refers to a fragment of an antibody containing two antigen-binding sites, generated by cleavage of the antibody molecule with the enzyme pepsin which cuts at the hinge region C-terminally to the inter-H-chain disulfide bond.

The term “Fc fragment” refers to the fragment of an antibody comprising the constant domain of its heavy chain.

The term “Fv fragment” refers to the fragment of an antibody comprising the variable domains of its heavy chain and light chain.

“Gene construct” refers to a nucleic acid, such as a vector, plasmid, viral genome or the like which includes a “coding sequence” for a polypeptide or which is otherwise transcribable to a biologically active RNA (e.g., antisense, decoy, ribozyme, etc), may be transfected into cells, e.g. in certain embodiments mammalian cells, and may cause expression of the coding sequence in cells transfected with the construct. The gene construct may include one or more regulatory elements operably linked to the coding sequence, as well as intronic sequences, polyadenylation sites, origins of replication, marker genes, etc.

The term “identity” refers to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 70%, 85%, 90%, 95%, 98% or 99% identity to specific polypeptides described herein and preferably exhibiting substantially the same functions, as well as polynucleotide encoding such polypeptides, are contemplated. Unless otherwise indicated a similarity score will be based on use of BLOSUM62. When BLASTP is used, the percent similarity is based on the BLASTP positives score and the percent sequence identity is based on the BLASTP identities score. BLASTP “Identities” shows the number and fraction of total residues in the high scoring sequence pairs which are identical; and BLASTP “Positives” shows the number and fraction of residues for which the alignment scores have positive values and which are similar to each other. Amino acid sequences having these degrees of identity or similarity or any intermediate degree of identity of similarity to the amino acid sequences disclosed herein are contemplated and encompassed by this disclosure. The polynucleotide sequences of similar polypeptides are deduced using the genetic code and may be obtained by conventional means, in particular by reverse translating its amino acid sequence using the genetic code.

The term “linker” is art-recognized and refers to a molecule or group of molecules connecting two compounds, such as two polypeptides. The linker may be comprised of a single linking molecule or may comprise a linking molecule and a spacer molecule, intended to separate the linking molecule and a compound by a specific distance.

The term “multivalent antibody” refers to an antibody or engineered antibody comprising more than one antigen recognition site. For example, a “bivalent” antibody has two antigen recognition sites, whereas a “tetravalent” antibody has four antigen recognition sites. The terms “monospecific”, “bispecific”, “trispecific”, “tetraspecific”, etc. refer to the number of different antigen recognition site specificities (as opposed to the number of antigen recognition sites) present in a multivalent antibody. For example, a “monospecific” antibody's antigen recognition sites all bind the same epitope. A “bispecific” antibody has at least one antigen recognition site that binds a first epitope and at least one antigen recognition site that binds a second epitope that is different from the first epitope. A “multivalent monospecific” antibody has multiple antigen recognition sites that all bind the same epitope. A “multivalent bispecific” antibody has multiple antigen recognition sites, some number of which bind a first epitope and some number of which bind a second epitope that is different from the first epitope.

The term “nucleic acid” refers to a natural or synthetic molecule comprising a single nucleotide or two or more nucleotides linked by a phosphate group at the 3′ position of one nucleotide to the 5′ end of another nucleotide. The nucleic acid is not limited by length, and thus the nucleic acid can include deoxyribonucleic acid (DNA) or ribonucleic acid (RNA).

The term “operably linked to” refers to the functional relationship of a nucleic acid with another nucleic acid sequence. Promoters, enhancers, transcriptional and translational stop sites, and other signal sequences are examples of nucleic acid sequences operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

The terms “peptide,” “protein,” and “polypeptide” are used interchangeably to refer to a natural or synthetic molecule comprising two or more amino acids linked by the carboxyl group of one amino acid to the alpha amino group of another.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The terms “polypeptide fragment” or “fragment”, when used in reference to a particular polypeptide, refers to a polypeptide in which amino acid residues are deleted as compared to the reference polypeptide itself, but where the remaining amino acid sequence is usually identical to that of the reference polypeptide. Such deletions may occur at the amino-terminus or carboxy-terminus of the reference polypeptide, or alternatively both. Fragments typically are at least about 5, 6, 8 or 10 amino acids long, at least about 14 amino acids long, at least about 20, 30, 40 or 50 amino acids long, at least about 75 amino acids long, or at least about 100, 150, 200, 300, 500 or more amino acids long. A fragment can retain one or more of the biological activities of the reference polypeptide. In various embodiments, a fragment may comprise an enzymatic activity and/or an interaction site of the reference polypeptide. In another embodiment, a fragment may have immunogenic properties.

The term “protein domain” refers to a portion of a protein, portions of a protein, or an entire protein showing structural integrity; this determination may be based on amino acid composition of a portion of a protein, portions of a protein, or the entire protein.

The term “single chain variable fragment or scFv” refers to an Fv fragment in which the heavy chain domain and the light chain domain are linked. One or more scFv fragments may be linked to other antibody fragments (such as the constant domain of a heavy chain or a light chain) to form antibody constructs having one or more antigen recognition sites.

A “spacer” as used herein refers to a peptide that joins the proteins comprising a fusion protein. Generally a spacer has no specific biological activity other than to join the proteins or to preserve some minimum distance or other spatial relationship between them. However, the constituent amino acids of a spacer may be selected to influence some property of the molecule such as the folding, net charge, or hydrophobicity of the molecule.

The term “specifically binds”, as used herein, when referring to a polypeptide (including antibodies) or receptor, refers to a binding reaction which is determinative of the presence of the protein or polypeptide or receptor in a heterogeneous population of proteins and other biologics. Thus, under designated conditions (e.g. immunoassay conditions in the case of an antibody), a specified ligand or antibody “specifically binds” to its particular “target” (e.g. an antibody specifically binds to an endothelial antigen) when it does not bind in a significant amount to other proteins present in the sample or to other proteins to which the ligand or antibody may come in contact in an organism. Generally, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰ 10⁻¹, M¹¹ M⁻¹, and 10¹² M⁻¹ or more) with that second molecule.

The term “specifically deliver” as used herein refers to the preferential association of a molecule with a cell or tissue bearing a particular target molecule or marker and not to cells or tissues lacking that target molecule. It is, of course, recognized that a certain degree of non-specific interaction may occur between a molecule and a non-target cell or tissue. Nevertheless, specific delivery, may be distinguished as mediated through specific recognition of the target molecule. Typically specific delivery results in a much stronger association between the delivered molecule and cells bearing the target molecule than between the delivered molecule and cells lacking the target molecule.

The term “subject” refers to any individual who is the target of administration or treatment. The subject can be a vertebrate, for example, a mammal. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The terms “transformation” and “transfection” mean the introduction of a nucleic acid, e.g., an expression vector, into a recipient cell including introduction of a nucleic acid to the chromosomal DNA of said cell.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “variant” refers to an amino acid or peptide sequence having conservative amino acid substitutions, non-conservative amino acid substitutions (i.e. a degenerate variant), substitutions within the wobble position of each codon (i.e. DNA and RNA) encoding an amino acid, amino acids added to the C-terminus of a peptide, or a peptide having 60%, 70%, 80%, 90%, 95%, 96%, 97%, 98%, 99% sequence identity to a reference sequence.

The term “vector” refers to a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element).

### Chimeric Antigen Receptors (CAR)

CARs generally incorporate an antigen recognition domain from the single-chain variable fragments (scFv) of a monoclonal antibody (mAb) with transmembrane signaling motifs involved in lymphocyte activation (Sadelain M, et al. Nat Rev Cancer 2003 3:35-45). A CAR is generally made up of three domains: an ectodomain, a transmembrane domain, and an endodomain. The ectodomain comprises the CD123-binding region and is responsible for antigen recognition. It also optionally contains a signal peptide (SP) so that the CAR can be glycosylated and anchored in the cell membrane of the immune effector cell. The transmembrane domain (TD), is as its name suggests, connects the ectodomain to the endodomain and resides within the cell membrane when expressed by a cell. The endodomain is the business end of the CAR that transmits an activation signal to the immune effector cell after antigen recognition. For example, the endodomain can contain an intracellular signaling domain (ISD) and optionally a co-stimulatory signaling region (CSR).

A “signaling domain (SD)” generally contains immunoreceptor tyrosine-based activation motifs (ITAMs) that activate a signaling cascade when the ITAM is phosphorylated. The term “co-stimulatory signaling region (CSR)” refers to intracellular signaling domains from costimulatory protein receptors, such as CD28, 41BB, and ICOS, that are able to enhance T-cell activation by T-cell receptors.

In some embodiments, the endodomain contains an SD or a CSR, but not both. In these embodiments, an immune effector cell containing the disclosed CAR is only activated if another CAR (or a T-cell receptor) containing the missing domain also binds its respective antigen.

Additional CAR constructs are described, for example, in Fresnak A D, et al. Engineered T cells: the promise and challenges of cancer immunotherapy. Nat Rev Cancer. 2016 Aug. 23; 16(9):566-81, which is incorporated by reference in its entirety for the teaching of these CAR models.

For example, the CAR can be a TRUCK, Universal CAR, Self-driving CAR, Armored CAR, Self-destruct CAR, Conditional CAR, Marked CAR, TenCAR, Dual CAR, or sCAR.

TRUCKs (T cells redirected for universal cytokine killing) co-express a chimeric antigen receptor (CAR) and an antitumor cytokine. Cytokine expression may be constitutive or induced by T cell activation. Targeted by CAR specificity, localized production of pro-inflammatory cytokines recruits endogenous immune cells to tumor sites and may potentiate an antitumor response.

Universal, allogeneic CAR T cells are engineered to no longer express endogenous T cell receptor (TCR) and/or major histocompatibility complex (MHC) molecules, thereby preventing graft-versus-host disease (GVHD) or rejection, respectively.

Self-driving CARs co-express a CAR and a chemokine receptor, which binds to a tumor ligand, thereby enhancing tumor homing.

CAR T cells engineered to be resistant to immunosuppression (Armored CARs) may be genetically modified to no longer express various immune checkpoint molecules (for example, cytotoxic T lymphocyte-associated antigen 4 (CTLA4) or programmed cell death protein 1 (PD1)), with an immune checkpoint switch receptor, or may be administered with a monoclonal antibody that blocks immune checkpoint signaling.

A self-destruct CAR may be designed using RNA delivered by electroporation to encode the CAR. Alternatively, inducible apoptosis of the T cell may be achieved based on ganciclovir binding to thymidine kinase in gene-modified lymphocytes or the more recently described system of activation of human caspase 9 by a small-molecule dimerizer.

A conditional CAR T cell is by default unresponsive, or switched ‘off’, until the addition of a small molecule to complete the circuit, enabling full transduction of both signal 1 and signal 2, thereby activating the CAR T cell. Alternatively, T cells may be engineered to express an adaptor-specific receptor with affinity for subsequently administered secondary antibodies directed at target antigen.

Marked CAR T cells express a CAR plus a tumor epitope to which an existing monoclonal antibody agent binds. In the setting of intolerable adverse effects, administration of the monoclonal antibody clears the CAR T cells and alleviates symptoms with no additional off-tumor effects.

A tandem CAR (TanCAR) T cell expresses a single CAR consisting of two linked single-chain variable fragments (scFvs) that have different affinities fused to intracellular co-stimulatory domain(s) and a CD3ζ domain. TanCAR T cell activation is achieved only when target cells co-express both targets.

A dual CAR T cell expresses two separate CARs with different ligand binding targets; one CAR includes only the CD3ζ domain and the other CAR includes only the co-stimulatory domain(s). Dual CAR T cell activation requires co-expression of both targets on the tumor.

A safety CAR (sCAR) consists of an extracellular scFv fused to an intracellular inhibitory domain. sCAR T cells co-expressing a standard CAR become activated only when encountering target cells that possess the standard CAR target but lack the sCAR target.

The antigen recognition domain of the disclosed CAR is usually an scFv. There are however many alternatives. An antigen recognition domain from native T-cell receptor (TCR) alpha and beta single chains have been described, as have simple ectodomains (e.g. CD4 ectodomain to recognize HIV infected cells) and more exotic recognition components such as a linked cytokine (which leads to recognition of cells bearing the cytokine receptor). In fact almost anything that binds a given target with high affinity can be used as an antigen recognition region.

The endodomain is the business end of the CAR that after antigen recognition transmits a signal to the immune effector cell, activating at least one of the normal effector functions of the immune effector cell. Effector function of a T cell, for example, may be cytolytic activity or helper activity including the secretion of cytokines. Therefore, the endodomain may comprise the “intracellular signaling domain” of a T cell receptor (TCR) and optional co-receptors. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal.

Cytoplasmic signaling sequences that regulate primary activation of the TCR complex that act in a stimulatory manner may contain signaling motifs which are known as immunoreceptor tyrosine-based activation motifs (ITAMs). Examples of ITAM containing cytoplasmic signaling sequences include those derived from CD8, CD3ζ, CD3δ, CD3γ, CD3ε, CD32 (Fc gamma RIla), DAP10, DAP12, CD79a, CD79b, FcγRIγ, FcγRIIIγ, FcεRIβ (FCERIB), and FcεRIγ (FCERIG).

In particular embodiments, the intracellular signaling domain is derived from CD3 zeta (CD3ζ) (TCR zeta, GenBank accno. BAG36664.1). T-cell surface glycoprotein CD3 zeta (CD3ζ) chain, also known as T-cell receptor T3 zeta chain or CD247 (Cluster of Differentiation 247), is a protein that in humans is encoded by the CD247 gene.

First-generation CARs typically had the intracellular domain from the CD3 chain, which is the primary transmitter of signals from endogenous TCRs. Second-generation CARs add intracellular signaling domains from various costimulatory protein receptors (e.g., CD28, 41BB, ICOS) to the endodomain of the CAR to provide additional signals to the T cell. Preclinical studies have indicated that the second generation of CAR designs improves the antitumor activity of T cells. More recent, third-generation CARs combine multiple signaling domains to further augment potency. T cells grafted with these CARs have demonstrated improved expansion, activation, persistence, and tumor-eradicating efficiency independent of costimulatory receptor/ligand interaction (Imai C, et al. Leukemia 2004 18:676-84; Maher J, et al. Nat Biotechnol 2002 20:70-5).

For example, the endodomain of the CAR can be designed to comprise the CD3ζ signaling domain by itself or combined with any other desired cytoplasmic domain(s) useful in the context of the CAR of the invention. For example, the cytoplasmic domain of the CAR can comprise a CD3ζ chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1BB (CD137), OX40, CD30, CD40, ICOS, lymphocyte function-associated antigen-1 (LFA-1), CD2, CD7, LIGHT, NKG2C, B7-H3, and a ligand that specifically binds with CD83, CD8, CD4, b2c, CD80, CD86, DAP10, DAP12, MyD88, BTNL3, and NKG2D. Thus, while the CAR is exemplified primarily with CD28 as the co-stimulatory signaling element, other costimulatory elements can be used alone or in combination with other co-stimulatory signaling elements.

In some embodiments, the CAR comprises a hinge sequence. A hinge sequence is a short sequence of amino acids that facilitates antibody flexibility (see, e.g., Woof et al., Nat. Rev. Immunol., 4(2): 89-99 (2004)). The hinge sequence may be positioned between the antigen recognition moiety (e.g., anti-CD123 scFv) and the transmembrane domain. The hinge sequence can be any suitable sequence derived or obtained from any suitable molecule. In some embodiments, for example, the hinge sequence is derived from a CD8a molecule or a CD28 molecule.

The transmembrane domain may be derived either from a natural or from a synthetic source. Where the source is natural, the domain may be derived from any membrane-bound or transmembrane protein. For example, the transmembrane region may be derived from (i.e. comprise at least the transmembrane region(s) of) the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, or CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R α, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, and PAG/Cbp. Alternatively the transmembrane domain may be synthetic, in which case it will comprise predominantly hydrophobic residues such as leucine and valine. In some cases, a triplet of phenylalanine, tryptophan and valine will be found at each end of a synthetic transmembrane domain. A short oligo- or polypeptide linker, such as between 2 and 10 amino acids in length, may form the linkage between the transmembrane domain and the endoplasmic domain of the CAR.

In some embodiments, the CAR has more than one transmembrane domain, which can be a repeat of the same transmembrane domain, or can be different transmembrane domains.

In some embodiments, the CAR is a multi-chain CAR, as described in WO2015/039523, which is incorporated by reference for this teaching. A multi-chain CAR can comprise separate extracellular ligand binding and signaling domains in different transmembrane polypeptides. The signaling domains can be designed to assemble in juxtamembrane position, which forms flexible architecture closer to natural receptors, that confers optimal signal transduction. For example, the multi-chain CAR can comprise a part of an FCERI alpha chain and a part of an FCERI beta chain such that the FCERI chains spontaneously dimerize together to form a CAR.

In some embodiments, the antigen binding agent is single chain variable fragment (scFv) antibody. The affinity/specificity of an scFv is driven in large part by specific sequences within complementarity determining regions (CDRs) in the heavy (V_{H}) and light (V_{L}) chain. Each V_{H} and V_{L} sequence will have three CDRs (CDR1, CDR2, CDR3).

In some embodiments, the antigen binding agent is derived from natural antibodies, such as monoclonal antibodies. In some cases, the antibody is human. In some cases, the antibody has undergone an alteration to render it less immunogenic when administered to humans. For example, the alteration comprises one or more techniques selected from the group consisting of chimerization, humanization, CDR-grafting, deimmunization, and mutation of framework amino acids to correspond to the closest human germline sequence.

Also disclosed are bi-specific CARs that target two tumor antigens. Also disclosed are CARs designed to work only in conjunction with another CAR that binds a different antigen, such as a tumor antigen. For example, in these embodiments, the endodomain of the disclosed CAR can contain only a signaling domain (SD) or a co-stimulatory signaling region (CSR), but not both. The second CAR (or endogenous T-cell) provides the missing signal if it is activated. For example, if the disclosed CAR contains an SD but not a CSR, then the immune effector cell containing this CAR is only activated if another CAR (or T-cell) containing a CSR binds its respective antigen. Likewise, if the disclosed CAR contains a CSR but not a SD, then the immune effector cell containing this CAR is only activated if another CAR (or T-cell) containing an SD binds its respective antigen.

### Transgenic T-Cell Receptor (TCR)

In some embodiments, the immune effector cells also express a transgenic TCRs, and can be used with adoptive cell transfer to target and kill cancers. The T-cell receptor (TCR) is a molecule found on the surface of T cells which is responsible for recognizing fragments of target antigen as peptides bound to major histocompatibility complex (MHC) molecules. The TCR is a heterodimer composed of two different protein chains. In humans, in 95% of T cells the TCR consists of an alpha (a) chain and a beta (b) chain (encoded by TRA and TRB, respectively), whereas in 5% of T cells the TCR consists of gamma and delta (g/d) chains (encoded by TRG and TRD, respectively).

Each chain is composed of two extracellular domains: a variable (V) region and a constant (C) region. The constant region is proximal to the cell membrane, followed by a transmembrane region and a short cytoplasmic tail, while the Variable region binds to the peptide/MHC complex. The variable domain of the TCR a-chain and b-chain each have three hypervariable or complementarity determining regions (CDRs). CDR3 is the main CDR responsible for recognizing processed antigen. The constant domain of the TCR consists of short connecting sequences in which a cysteine residue forms disulphide bonds, which form a link between the two chains. When the TCR engages with antigenic peptide and MHC (peptide/MHC), the T lymphocyte is activated through signal transduction. In contrast to conventional antibody-directed target antigens, antigens recognized by the TCR can include the entire array of potential intracellular proteins, which are processed and delivered to the cell surface as a peptide/MHC complex.

It is possible to engineer cells to express heterologous (i.e. non-native) TCR molecules by artificially introducing the TRA and TRB genes; or TRG and TRD genes into the cell using a vector. Such heterologous TCRs may also be referred to herein as transgenic TCRs. For example, the genes for genetically modified TCRs may be reintroduced into autologous T cells and transferred back into patients for T cell adoptive therapies. In some embodiments, the transgenic TCR is a recombinant TCR.

### Tumor Antigens

Tumor antigens are proteins that are produced by tumor cells that elicit an immune response, particularly T-cell mediated immune responses. The additional antigen binding domain can be an antibody or a natural ligand of the tumor antigen. The selection of the additional antigen binding domain will depend on the particular type of cancer to be treated. Tumor antigens are well known in the art and include, for example, a glioma-associated antigen, carcinoembryonic antigen (CEA), EGFRvIII, IL-11Ra, IL-13Ra, EGFR, FAP, B7H3, Kit, CA LX, CS-1, MUC1, BCMA, bcr-abl, HER2, β-human chorionic gonadotropin, alphafetoprotein (AFP), ALK, CD19, TIM3, cyclin BI, lectin-reactive AFP, Fos-related antigen 1, ADRB3, thyroglobulin, EphA2, RAGE-1, RUI, RU2, SSX2, AKAP-4, LCK, OY-TESI, PAX5, SART3, CLL-1, fucosyl GM1, GloboH, MN-CA IX, EPCAM, EVT6-AML, TGS5, human telomerase reverse transcriptase, plysialic acid, PLAC1, RUI, RU2 (AS), intestinal carboxyl esterase, lewisY, sLe, LY6K, mut hsp70-2, M-CSF, MYCN, RhoC, TRP-2, CYPIBI, BORIS, prostase, prostate-specific antigen (PSA), PAX3, PAP, NY-ESO-1, LAGE-Ia, LMP2, NCAM, p53, p53 mutant, Ras mutant, gplOO, prostein, OR51E2, PANX3, PSMA, PSCA, Her2/neu, hTERT, HMWMAA, HAVCR1, VEGFR2, PDGFR-beta, survivin and telomerase, legumain, HPV E6,E7, sperm protein 17, SSEA-4, tyrosinase, TARP, WT1, prostate-carcinoma tumor antigen-1 (PCTA-1), ML-IAP, MAGE, MAGE-A1,MAD-CT-1, MAD-CT-2, MelanA/MART 1, XAGE1, ELF2M, ERG (TMPRSS2 ETS fusion gene), NA17, neutrophil elastase, sarcoma translocation breakpoints, NY-BR-1, ephnnB2, CD20, CD22, CD24, CD30, TIM3, CD38, CD44v6, CD97, CD171, CD179a, androgen receptor, FAP, insulin growth factor (IGF)-I, IGFII, IGF-I receptor, GD2, o-acetyl-GD2, GD3, GM3, GPRC5D, GPR20, CXORF61, folate receptor (FRa), folate receptor beta, ROR1, Flt3, TAG72, TN Ag, T_{reg} 2, TEM1, TEM7R, CLDN6, TSHR, UPK2, and mesothelin. In a preferred embodiment, the tumor antigen is selected from the group consisting of folate receptor (FRa), mesothelin, EGFRvIII, IL-13Ra, CD123, CD19, TIM3, BCMA, GD2, CLL-1, CA-IX, MUCI, HER2, and any combination thereof.

Non-limiting examples of tumor antigens include the following: Differentiation antigens such as tyrosinase, TRP-1, TRP-2 and tumor-specific multilineage antigens such as MAGE-1, MAGE-3, BAGE, GAGE-1, GAGE-2, pi 5; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, pI85erbB2, pI80erbB-3, c-met, nm-23H1, PSA, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCASI, SDCCAG1 6, TA-90\Mac-2 binding protein\cyclophilm C-associated protein, TAAL6, TAG72, TLP, TPS, GPC3, MUC16, LMP1, EBMA-1, BARF-1, CS1, CD319, HER1, B7H6, L1CAM, IL6, and MET.

### Nucleic Acids and Vectors

Also disclosed are polynucleotides and polynucleotide vectors encoding the disclosed transcription factors, dominant negative transcription factors, CARs, or any combination thereof.

Nucleic acid sequences encoding the disclosed transcription factors, dominant negative transcription factors, CARs, and regions thereof, can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

Expression of nucleic acids is typically achieved by operably linking a nucleic acid encoding to a promoter, and incorporating the construct into an expression vector. Typical cloning vectors contain transcription and translation terminators, initiation sequences, and promoters useful for regulation of the expression of the desired nucleic acid sequence.

The disclosed nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. In some embodiments, the polynucleotide vectors are lentiviral or retroviral vectors.

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, MND (myeloproliferative sarcoma virus) promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. The promoter can alternatively be an inducible promoter. Examples of inducible promoters include, but are not limited to a metallothionine promoter, a glucocorticoid promoter, a progesterone promoter, and a tetracycline promoter.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another.

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene. Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York).

Biological methods for introducing a polynucleotide of interest into a host cell include the use of DNA and RNA vectors. Viral vectors, and especially retroviral vectors, have become the most widely used method for inserting genes into mammalian, e.g., human cells.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes. Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DM PG”) and other lipids may be obtained from Avanti Polar Lipids, Inc, (Birmingham, Ala.).

### Immune Effector Cells

The disclosed immune effector cells are preferably obtained from the subject to be treated (i.e. are autologous). However, in some embodiments, immune effector cell lines or donor effector cells (allogeneic) are used. Immune effector cells can be obtained from a number of sources, including peripheral blood mononuclear cells, bone marrow, lymph node tissue, cord blood, thymus tissue, tissue from a site of infection, ascites, pleural effusion, spleen tissue, and tumors. Immune effector cells can be obtained from blood collected from a subject using any number of techniques known to the skilled artisan, such as Ficoll™ separation. For example, cells from the circulating blood of an individual may be obtained by apheresis. In some embodiments, immune effector cells are isolated from peripheral blood lymphocytes by lysing the red blood cells and depleting the monocytes, for example, by centrifugation through a PERCOLL™ gradient or by counterflow centrifugal elutriation. A specific subpopulation of immune effector cells can be further isolated by positive or negative selection techniques. For example, immune effector cells can be isolated using a combination of antibodies directed to surface markers unique to the positively selected cells, e.g., by incubation with antibody-conjugated beads for a time period sufficient for positive selection of the desired immune effector cells. Alternatively, enrichment of immune effector cells population can be accomplished by negative selection using a combination of antibodies directed to surface markers unique to the negatively selected cells.

In some embodiments, the immune effector cells comprise any leukocyte involved in defending the body against infectious disease and foreign materials. For example, the immune effector cells can comprise lymphocytes, monocytes, macrophages, dentritic cells, mast cells, neutrophils, basophils, eosinophils, or any combinations thereof. For example, the immune effector cells can comprise T lymphocytes.

T cells or T lymphocytes can be distinguished from other lymphocytes, such as B cells and natural killer cells (NK cells), by the presence of a T-cell receptor (TCR) on the cell surface. They are called T cells because they mature in the thymus (although some also mature in the tonsils). There are several subsets of T cells, each with a distinct function.

T helper cells (T_{H} cells) assist other white blood cells in immunologic processes, including maturation of B cells into plasma cells and memory B cells, and activation of cytotoxic T cells and macrophages. These cells are also known as CD4+ T cells because they express the CD4 glycoprotein on their surface. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including T_{H}1, T_{H}2, T_{H}3, T_{H}17, T_{H}9, or T_{FH}, which secrete different cytokines to facilitate a different type of immune response.

Cytotoxic T cells (T_{C} cells, or CTLs) destroy virally infected cells and tumor cells, and are also implicated in transplant rejection. These cells are also known as CD8⁺ T cells since they express the CD8 glycoprotein at their surface. These cells recognize their targets by binding to antigen associated with MHC class I molecules, which are present on the surface of all nucleated cells. Through IL-10, adenosine and other molecules secreted by regulatory T cells, the CD8+ cells can be inactivated to an anergic state, which prevents autoimmune diseases.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory cells may be either CD4⁺ or CD8⁺. Memory T cells typically express the cell surface protein CD45RO.

Regulatory T cells (T_{reg} cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus. Two major classes of CD4⁺ T_{reg} cells have been described—naturally occurring T_{reg} cells and adaptive T_{reg} cells.

Natural killer T (NKT) cells (not to be confused with natural killer (NK) cells) bridge the adaptive immune system with the innate immune system. Unlike conventional T cells that recognize peptide antigens presented by major histocompatibility complex (MHC) molecules, NKT cells recognize glycolipid antigen presented by a molecule called CD1d.

In some embodiments, the T cells comprise a mixture of CD4+ cells. In other embodiments, the T cells are enriched for one or more subsets based on cell surface expression. For example, in some cases, the T comprise are cytotoxic CD8⁺ T lymphocytes. In some embodiments, the T cells comprise γδ T cells, which possess a distinct T-cell receptor (TCR) having one γ chain and one δ chain instead of α and β chains.

Natural-killer (NK) cells are CD56⁺CD3⁻ large granular lymphocytes that can kill virally infected and transformed cells, and constitute a critical cellular subset of the innate immune system (Godfrey J, et al. Leuk Lymphoma 2012 53:1666-1676). Unlike cytotoxic CD8⁺ T lymphocytes, NK cells launch cytotoxicity against tumor cells without the requirement for prior sensitization, and can also eradicate MHC-I-negative cells (Narni-Mancinelli E, et al. Int Immunol 2011 23:427-431). NK cells are safer effector cells, as they may avoid the potentially lethal complications of cytokine storms (Morgan R A, et al. Mol Ther 2010 18:843-851), tumor lysis syndrome (Porter D L, et al. N Engl J Med 2011 365:725-733), and on-target, off-tumor effects.

In some embodiments, the immune effector cells are tumor infiltrating lymphocytes (TILs) isolated from the subject and expanded ex vivo. In Adoptive T cell transfer therapy, TILs are expanded ex vivo from surgically resected tumors that have been cut into small fragments or from single cell suspensions isolated from the tumor fragments. Multiple individual cultures are established, grown separately and assayed for specific tumor recognition. TILs can be expanded over the course of a few weeks with a high dose of IL-2 in 24-well plates. Selected TIL lines that presented best tumor reactivity can then be further expanded in a “rapid expansion protocol” (REP), which uses anti-CD3 activation for a typical period of two weeks. The final post-REP can be infused back into the patient. The process can also involve a preliminary chemotherapy regimen to deplete endogenous lymphocytes in order to provide the adoptively transferred TILs with enough access to surround the tumor sites.

In some embodiments, the “immune effector cells” are progenitor cells or stem cells with the potential to become lymphocytes. For example, in some embodiments, the cells are induced pluripotent stem cells (iPSCs). iPSCs are typically derived by introducing products of specific sets of pluripotency-associated genes, or “reprogramming factors”, into a given cell type. The original set of reprogramming factors (also dubbed Yamanaka factors) are the transcription factors Oct4 (Pou5f1), Sox2, cMyc, and Klf4. While this combination is most conventional in producing iPSCs, each of the factors can be functionally replaced by related transcription factors, miRNAs, small molecules, or even non-related genes such as lineage specifiers.

### Therapeutic Methods

Immune effector cells expressing the disclosed transcription factors and/or dominant negative constructs can elicit an anti-tumor immune response against tumors. The anti-tumor immune response elicited by the disclosed recombinant immune effector cells may be an active or a passive immune response. In addition, the immune response may be part of an adoptive immunotherapy approach in which recombinant immune effector cells induce an enhanced immune response.

Adoptive transfer of immune effector cells is a promising anti-cancer therapeutic. Following the collection of a patient's immune effector cells, the cells may be genetically engineered to express the disclosed transcription factors and/or dominant negative constructs, and optionally CARs, then infused back into the patient.

The disclosed recombinant immune effector cells may be administered either alone, or as a pharmaceutical composition in combination with diluents and/or with other components such as IL-2, IL-15, or other cytokines or cell populations. Briefly, pharmaceutical compositions may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions for use in the disclosed methods are in some embodiments formulated for intravenous administration. Pharmaceutical compositions may be administered in any manner appropriate treat MM. The quantity and frequency of administration will be determined by such factors as the condition of the patient, and the severity of the patient's disease, although appropriate dosages may be determined by clinical trials.

When “an immunologically effective amount”, “an anti-tumor effective amount”, “an tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, such as 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In certain embodiments, it may be desired to administer activated T cells to a subject and then subsequently re-draw blood (or have an apheresis performed), activate T cells therefrom according to the disclosed methods, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Using this multiple blood draw/multiple reinfusion protocol may serve to select out certain populations of T cells.

The administration of the disclosed compositions may be carried out in any convenient manner, including by injection, transfusion, or implantation. The compositions described herein may be administered to a patient subcutaneously, intradermally, intratumorally, intranodally, intramedullary, intramuscularly, by intravenous (i.v.) injection, or intraperitoneally. In some embodiments, the disclosed compositions are administered to a patient by intradermal or subcutaneous injection. In some embodiments, the disclosed compositions are administered by i.v. injection. The compositions may also be injected directly into a tumor, lymph node, or site of infection.

In certain embodiments, the disclosed recombinant immune effector cells are administered to a patient in conjunction with (e.g., before, simultaneously or following) any number of relevant treatment modalities, including but not limited to thalidomide, dexamethasone, bortezomib, and lenalidomide. In further embodiments, the CAR-modified immune effector cells may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. In some embodiments, the CAR-modified immune effector cells are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAM PATH. In another embodiment, the cell compositions of the present invention are administered following B-cell ablative therapy such as agents that react with CD20, e.g., Rituxan. For example, in some embodiments, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

The cancer of the disclosed methods can be any cell in a subject undergoing unregulated growth, invasion, or metastasis. In some aspects, the cancer can be any neoplasm or tumor for which radiotherapy is currently used. Alternatively, the cancer can be a neoplasm or tumor that is not sufficiently sensitive to radiotherapy using standard methods. Thus, the cancer can be a sarcoma, lymphoma, leukemia, carcinoma, blastoma, or germ cell tumor. A representative but non-limiting list of cancers that the disclosed compositions can be used to treat include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, endometrial cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

The disclosed cells can be used in combination with any compound, moiety or group which has a cytotoxic or cytostatic effect. Drug moieties include chemotherapeutic agents, which may function as microtubulin inhibitors, mitosis inhibitors, topoisomerase inhibitors, or DNA intercalators, and particularly those which are used for cancer therapy.

The disclosed cells can be used in combination with a checkpoint inhibitor. The two known inhibitory checkpoint pathways involve signaling through the cytotoxic T-lymphocyte antigen-4 (CTLA-4) and programmed-death 1 (PD-1) receptors. These proteins are members of the CD28-B7 family of cosignaling molecules that play important roles throughout all stages of T cell function. The PD-1 receptor (also known as CD279) is expressed on the surface of activated T cells. Its ligands, PD-L1 (B7-H1; CD274) and PD-L2 (B7-DC; CD273), are expressed on the surface of APCs such as dendritic cells or macrophages. PD-L1 is the predominant ligand, while PD-L2 has a much more restricted expression pattern. When the ligands bind to PD-1, an inhibitory signal is transmitted into the T cell, which reduces cytokine production and suppresses T-cell proliferation. Checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

Human monoclonal antibodies to programmed death 1 (PD-1) and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

In some embodiments, the PDL1 inhibitor comprises an antibody that specifically binds PDL1, such as BMS-936559 (Bristol-Myers Squibb) or MPDL3280A (Roche). In some embodiments, the PD1 inhibitor comprises an antibody that specifically binds PD1, such as lambrolizumab (Merck), nivolumab (Bristol-Myers Squibb), or MED14736 (AstraZeneca). Human monoclonal antibodies to PD-1 and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

The disclosed cells can be used in combination with other cancer immunotherapies. There are two distinct types of immunotherapy: passive immunotherapy uses components of the immune system to direct targeted cytotoxic activity against cancer cells, without necessarily initiating an immune response in the patient, while active immunotherapy actively triggers an endogenous immune response. Passive strategies include the use of the monoclonal antibodies (mAbs) produced by B cells in response to a specific antigen. The development of hybridoma technology in the 1970s and the identification of tumor-specific antigens permitted the pharmaceutical development of mAbs that could specifically target tumor cells for destruction by the immune system. Thus far, mAbs have been the biggest success story for immunotherapy; the top three best-selling anticancer drugs in 2012 were mAbs. Among them is rituximab (Rituxan, Genentech), which binds to the CD20 protein that is highly expressed on the surface of B cell malignancies such as non-Hodgkin's lymphoma (NHL). Rituximab is approved by the FDA for the treatment of NHL and chronic lymphocytic leukemia (CLL) in combination with chemotherapy. Another important mAb is trastuzumab (Herceptin; Genentech), which revolutionized the treatment of HER2 (human epidermal growth factor receptor 2)-positive breast cancer by targeting the expression of HER2.

Generating optimal “killer” CD8 T cell responses also requires T cell receptor activation plus co-stimulation, which can be provided through ligation of tumor necrosis factor receptor family members, including OX40 (CD134) and 4-1BB (CD137). OX40 is of particular interest as treatment with an activating (agonist) anti-OX40 mAb augments T cell differentiation and cytolytic function leading to enhanced anti-tumor immunity against a variety of tumors.

In some embodiments, such an additional therapeutic agent may be selected from an antimetabolite, such as methotrexate, 6-mercaptopurine, 6-thioguanine, cytarabine, fludarabine, 5-fluorouracil, decarbazine, hydroxyurea, asparaginase, gemcitabine or cladribine.

In some embodiments, such an additional therapeutic agent may be selected from an alkylating agent, such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin.

In some embodiments, such an additional therapeutic agent is a targeted agent, such as ibrutinib or idelalisib.

In some embodiments, such an additional therapeutic agent is an epigenetic modifier such as azacitdine or vidaza.

In some embodiments, such an additional therapeutic agent may be selected from an anti-mitotic agent, such as taxanes, for instance docetaxel, and paclitaxel, and vinca alkaloids, for instance vindesine, vincristine, vinblastine, and vinorelbine.

In some embodiments, such an additional therapeutic agent may be selected from a topoisomerase inhibitor, such as topotecan or irinotecan, or a cytostatic drug, such as etoposide and teniposide.

In some embodiments, such an additional therapeutic agent may be selected from a growth factor inhibitor, such as an inhibitor of ErbBI (EGFR) (such as an EGFR antibody, e.g. zalutumumab, cetuximab, panitumumab or nimotuzumab or other EGFR inhibitors, such as gefitinib or erlotinib), another inhibitor of ErbB2 (HER2/neu) (such as a HER2 antibody, e.g. trastuzumab, trastuzumab-DM I or pertuzumab) or an inhibitor of both EGFR and HER2, such as lapatinib).

In some embodiments, such an additional therapeutic agent may be selected from a tyrosine kinase inhibitor, such as imatinib (Glivec, Gleevec ST1571) or lapatinib.

In some embodiments, the disclosed cells are administered in combination with ofatumumab, zanolimumab, daratumumab, ranibizumab, nimotuzumab, panitumumab, hu806, daclizumab (Zenapax), basiliximab (Simulect), infliximab (Remicade), adalimumab (Humira), natalizumab (Tysabri), omalizumab (Xolair), efalizumab (Raptiva), and/or rituximab.

In some embodiments, the disclosed cells are administered in combination with an anti-cancer cytokine, chemokine, or combination thereof. Examples of suitable cytokines and growth factors include IFNγ, IL-2, IL-4, IL-6, IL-7, IL-10, IL-12, IL-13, IL-15, IL-18, IL-23, IL-24, IL-27, IL-28a, IL-28b, IL-29, KGF, IFNa (e.g., INFa2b), IFN, GM-CSF, CD40L, Flt3 ligand, stem cell factor, ancestim, and TNFa. Suitable chemokines may include Glu-Leu-Arg (ELR)− negative chemokines such as IP-10, MCP-3, MIG, and SDF-Ia from the human CXC and C-C chemokine families. Suitable cytokines include cytokine derivatives, cytokine variants, cytokine fragments, and cytokine fusion proteins.

In some embodiments, the disclosed cells are administered in combination with a cell cycle control/apoptosis regulator (or “regulating agent”). A cell cycle control/apoptosis regulator may include molecules that target and modulate cell cycle control/apoptosis regulators such as (i) cdc-25 (such as NSC 663284), (ii) cyclin-dependent kinases that overstimulate the cell cycle (such as flavopiridol (L868275, HMR1275), 7-hydroxystaurosporine (UCN-01, KW-2401), and roscovitine (R-roscovitine, CYC202)), and (iii) telomerase modulators (such as BIBR1532, SOT-095, GRN163 and compositions described in for instance U.S. Pat. Nos. 6,440,735 and 6,713,055). Non-limiting examples of molecules that interfere with apoptotic pathways include TNF-related apoptosis-inducing ligand (TRAIL)/apoptosis-2 ligand (Apo-2L), antibodies that activate TRAIL receptors, IFNs, and anti-sense Bcl-2.

In some embodiments, the disclosed cells are administered in combination with a hormonal regulating agent, such as agents useful for anti-androgen and anti-estrogen therapy. Examples of such hormonal regulating agents are tamoxifen, idoxifene, fulvestrant, droloxifene, toremifene, raloxifene, diethylstilbestrol, ethinyl estradiol/estinyl, an antiandrogene (such as flutaminde/eulexin), a progestin (such as such as hydroxyprogesterone caproate, medroxy-progesterone/provera, megestrol acepate/megace), an adrenocorticosteroid (such as hydrocortisone, prednisone), luteinizing hormone-releasing hormone (and analogs thereof and other LHRH agonists such as buserelin and goserelin), an aromatase inhibitor (such as anastrazole/arimidex, aminoglutethimide/cytraden, exemestane) or a hormone inhibitor (such as octreotide/sandostatin).

In some embodiments, the disclosed cells are administered in combination with an anti-cancer nucleic acid or an anti-cancer inhibitory RNA molecule.

Combined administration, as described above, may be simultaneous, separate, or sequential. For simultaneous administration the agents may be administered as one composition or as separate compositions, as appropriate.

In some embodiments, the disclosed cells are administered in combination with radiotherapy. Radiotherapy may comprise radiation or associated administration of radiopharmaceuticals to a patient is provided. The source of radiation may be either external or internal to the patient being treated (radiation treatment may, for example, be in the form of external beam radiation therapy (EBRT) or brachytherapy (BT)). Radioactive elements that may be used in practicing such methods include, e.g., radium, cesium-137, iridium-192, americium-241, gold-198, cobalt-57, copper-67, technetium-99, iodide-123, iodide-131, and indium-111.

In some embodiments, the disclosed cells are administered in combination with surgery.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1

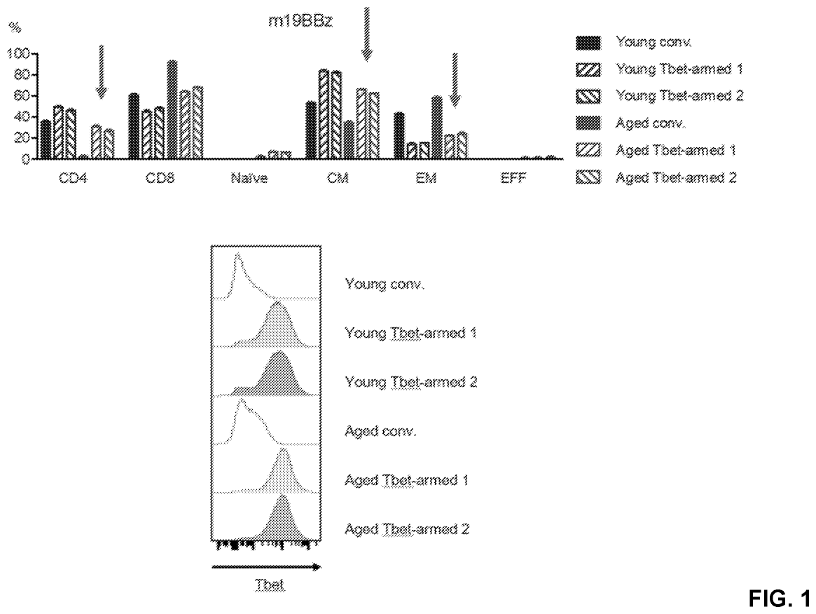

FIG. 1 shows effect of T-bet expression on m19BBz CAR-T cells.

### Example 2

The following are examples of transcription factor and dominant negative transcription factor constructs inserted into a SFG-h1928 CAR construct separated by a P2A cleavage site

BglII to NcoI in SFG-h1928z:
(SEQ ID NO: 7)
AGATCTTATATGGGGCACCCCCGCCCCTTGTAAACTTCCCTGACCCTGACATGACAAGAGTTACTAACAGCCCCTCTCTCCAAG
CTCACTTACAGGCTCTCTACTTAGTCCAGCACGAAGTCTGGAGACCTCTGGCGGCAGCCTACCAAGAACAACTGGACCGACCGG
TGGTACCTCACCCTTACCGAGTCGGCGACACAGTGTGGGTCCGCCGACACCAGACTAAGAACCTAGAACCTCGCTGGAAAGGAC
CTTACACAGTCCTGCTGACCACCCCCACCGCCCTCAAAGTAGACGGCATCGCAGCTTGGATACACGCCGCCCACCATGG.
SFGT bet-P2A-h1928z:
(SEQ ID NO: 8)
AGATCTTATATGGGGCACCCCCGCCCCTTGTAAACTTCCCTGACCCTGACATGACAAGAGTTACTAACAGCCCCTCTCTCCAAG
CTCACTTACAGGCTCTCTACTTAGTCCAGCACGAAGTCTGGAGACCTCTGGCGGCAGCCTACCAAGAACAACTGGACCGACCGG
TGGTACCTCACCCTTACCGAGTCGGCGACACAGTGTGGGTCCGCCGACACCAGACTAAGAACCTAGAACCTCGCTGGAAAGGAC
CTTACACAGTCCTGCTGACCACCCCCACCGCCCTCAAAGTAGACGGCATCGCAGCTTGGATACACGCCGCCCAATGGGCATTGT
GGAACCGGGCTGCGGCGATATGCTGACCGGCACCGAACCGATGCCGGGCAGCGATGAAGGCCGCGCGCCGGGCGCGGATCCGCA
GCATCGCTATTTTTATCCGGAACCGGGCGCGCAGGATGCGGATGAACGCCGCGGCGGCGGCAGCCTGGGCAGCCCGTATCCGGG
CGGCGCGCTGGTGCCGGCGCCGCCGAGCCGCTTTCTGGGCGCGTATGCGTATCCGCCGCGCCCGCAGGCGGCGGGCTTTCCGGG
CGCGGGCGAAAGCTTTCCGCCGCCGGCGGATGCGGAAGGCTATCAGCCGGGCGAAGGCTATGCGGCGCCGGATCCGCGCGCGGG
CCTGTATCCGGGCCCGCGCGAAGATTATGCGCTGCCGGCGGGCCTGGAAGTGAGCGGCAAACTGCGCGTGGCGCTGAACAACCA
TCTGCTGTGGAGCAAATTTAACCAGCATCAGACCGAAATGATTATTACCAAACAGGGCCGCCGCATGTTTCCGTTTCTGAGCTT
TACCGTGGCGGGCCTGGAACCGACCAGCCATTATCGCATGTTTGTGGATGTGGTGCTGGTGGATCAGCATCATTGGCGCTATCA
GAGCGGCAAATGGGTGCAGTGCGGCAAAGCGGAAGGCAGCATGCCGGGCAACCGCCTGTATGTGCATCCGGATAGCCCGAACAC
CGGCGCGCATTGGATGCGCCAGGAAGTGAGCTTTGGCAAACTGAAACTGACCAACAACAAAGGCGCGAGCAACAACGTGACCCA
GATGATTGTGCTGCAGAGCCTGCATAAATATCAGCCGCGCCTGCATATTGTGGAAGTGAACGATGGCGAACCGGAAGCGGCGTG
CAACGCGAGCAACACCCATATTTTTACCTTTCAGGAAACCCAGTTTATTGCGGTGACCGCGTATCAGAACGCGGAAATTACCCA
GCTGAAAATTGATAACAACCCGTTTGCGAAAGGCTTTCGCGAAAACTTTGAAAGCATGTATACCAGCGTGGATACCAGCATTCC
GAGCCCGCCGGGCCCGAACTGCCAGTTTCTGGGCGGCGATCATTATAGCCCGCTGCTGCCGAACCAGTATCCGGTGCCGAGCCG
CTTTTATCCGGATCTGCCGGGCCAGGCGAAAGATGTGGTGCCGCAGGCGTATTGGCTGGGCGCGCCGCGCGATCATAGCTATGA
AGCGGAATTTCGCGCGGTGAGCATGAAACCGGCGTTTCTGCCGAGCGCGCCGGGCCCGACCATGAGCTATTATCGCGGCCAGGA
AGTGCTGGCGCCGGGCGCGGGCTGGCCGGTGGCGCCGCAGTATCCGCCGAAAATGGGCCCGGCGAGCTGGTTTCGCCCGATGCG
CACCCTGCCGATGGAACCGGGCCCGGGCGGCAGCGAAGGCCGCGGCCCGGAAGATCAGGGCCCGCCGCTGGTGTGGACCGAAAT
TGCGCCGATTCGCCCGGAAAGCAGCGATAGCGGCCTGGGCGAAGGCGATAGCAAACGCCGCCGCGTGAGCCCGTATCCGAGCAG
CGGCGATAGCAGCAGCCCGGCGGGCGCGCCGAGCCCGTTTGATAAAGAAGCGGAAGGCCAGTTTTATAACTATTTTCCGAACGG
CAGCGGCGCGACCAACTTTAGCCTGCTGAAACAGGCGGGCGATGTGGAAGAAAACCCGGGCCCGCCATGG.
SFG DN Tbet-P2A-h1928z:
(SEQ ID NO: 9)
AGATCTTATATGGGGCACCCCCGCCCCTTGTAAACTTCCCTGACCCTGACATGACAAGAGTTACTAACAGCCCCTCTCTCCAAG
CTCACTTACAGGCTCTCTACTTAGTCCAGCACGAAGTCTGGAGACCTCTGGCGGCAGCCTACCAAGAACAACTGGACCGACCGG
TGGTACCTCACCCTTACCGAGTCGGCGACACAGTGTGGGTCCGCCGACACCAGACTAAGAACCTAGAACCTCGCTGGAAAGGAC
CTTACACAGTCCTGCTGACCACCCCCACCGCCCTCAAAGTAGACGGCATCGCAGCTTGGATACACGCCGCCCAATGGGCATTGT
GGAACCGGGCTGCGGCGATATGCTGACCGGCACCGAACCGATGCCGGGCAGCGATGAAGGCCGCGCGCCGGGCGCGGATCCGCA
GCATCGCTATTTTTATCCGGAACCGGGCGCGCAGGATGCGGATGAACGCCGCGGCGGCGGCAGCCTGGGCAGCCCGTATCCGGG
CGGCGCGCTGGTGCCGGCGCCGCCGAGCCGCTTTCTGGGCGCGTATGCGTATCCGCCGCGCCCGCAGGCGGCGGGCTTTCCGGG
CGCGGGCGAAAGCTTTCCGCCGCCGGCGGATGCGGAAGGCTATCAGCCGGGCGAAGGCTATGCGGCGCCGGATCCGCGCGCGGG
CCTGTATCCGGGCCCGCGCGAAGATTATGCGCTGCCGGCGGGCCTGGAAGTGAGCGGCAAACTGCGCGTGGCGCTGAACAACCA
TCTGCTGTGGAGCAAATTTAACCAGCATCAGACCGAAATGATTATTACCAAACAGGGCCGCCGCATGTTTCCGTTTCTGAGCTT
TACCGTGGCGGGCCTGGAACCGACCAGCCATTATCGCATGTTTGTGGATGTGGTGCTGGTGGATCAGCATCATTGGCGCTATCA
GAGCGGCAAATGGGTGCAGTGCGGCAAAGCGGAAGGCAGCATGCCGGGCAACCGCCTGTATGTGCATCCGGATAGCCCGAACAC
CGGCGCGCATTGGATGCGCCAGGAAGTGAGCTTTGGCAAACTGAAACTGACCAACAACAAAGGCGCGAGCAACAACGTGACCCA
GATGATTGTGCTGCAGAGCCTGCATAAATATCAGCCGCGCCTGCATATTGTGGAAGTGAACGATGGCGAACCGGAAGCGGCGTG
CAACGCGAGCAACACCCATATTTTTACCTTTCAGGAAACCCAGTTTATTGCGGTGACCGCGTATCAGAACGCGGAAATTACCCA
GCTGAAAATTGATAACAACCCGTTTGCGAAAGGCTTTCGCGAAAACTTTGAAAGCATGTATACCAGCGTGGATACCAGCATTCC
GAGCCCGCCGGGCCCGAACTGCCAGTTTCTGGGCGGCGATCATTATAGCCCGCTGCTGCCGAACCAGTATCCGGTGCCGAGCCG
CTTTTATCCGGATATGGCGCTGGAAGATCGCTGCAGCCCGCAGAGCGCGCCGAGCCCGATTACCCTGCAGATGCAGCATCTGCA
TCATCAGCAGCAGCAGCAGCAGCAGCAGCAGCAGCAGATGCAGCATCTGCATCAGCTGCAGCAGCTGCAGCAGCTGCATCAGCA
GCAGCTGGCGGCGGGCGTGTTTCATCATCCGGCGATGGCGTTTGATGCGGCGGCGGCGGCGGCGGCGGCGGCGGCGGCGGCGGC
GGCGCATGCGCATGCGGCGGCGCTGCAGCAGCGCCTGAGCGGCAGCGGCAGCCCGGCGAGCTGCAGCACCCCGGCGAGCAGCAC
CCCGCTGACCATTAAAGAAGAAGAAAGCGATAGCGTGATTGGCGATATGAGCTTTCATAACCAGACCCATACCACCAACGAAGA
AGAAGAAGCGGAAGAAGATGATGATATTGATGTGGATGTGGATGATACCAGCGCGGGCGGCCGCCTGCCGCCGCCGGCGCATCA
GCAGCAGAGCACCGCGAAACCGAGCCTGGCGTTTAGCATTAGCAACATTCTGAGCGATCGCTTTGGCGATGTGCAGAAACCGGG
CAAAAGCATTGAAAACCAGGCGAGCATTTTTCGCCCGTTTGAAGCGAACCGCAGCCAGACCGCGACCCCGAGCGCGTTTACCCG
CGTGGATCTGCTGGAATTTAGCCGCCAGCAGCAGGCGGCGGCGGCGGCGGCGACCGCGGCGATGATGCTGGAACGCGCGAACTT
TCTGAACTGCTTTAACCCGGCGGCGTATCCGCGCATTCATGAAGAAATTGTGCAGAGCCGCCTGCGCCGCAGCGCGGCGAACGC
GGTGATTCCGCCGCCGATGAGCAGCAAAATGAGCGATGCGAACCCGGAAAAAAGCGCGCTGGGCAGCGGCAGCGGCGCGACCAA
CTTTAGCCTGCTGAAACAGGCGGGCGATGTGGAAGAAAACCCGGGCCCGCCATGG.
SFG Eomes-P2A-h1928z:
(SEQ ID NO: 10)
AGATCTTATATGGGGCACCCCCGCCCCTTGTAAACTTCCCTGACCCTGACATGACAAGAGTTACTAACAGCCCCTCTCTCCAAG
CTCACTTACAGGCTCTCTACTTAGTCCAGCACGAAGTCTGGAGACCTCTGGCGGCAGCCTACCAAGAACAACTGGACCGACCGG
TGGTACCTCACCCTTACCGAGTCGGCGACACAGTGTGGGTCCGCCGACACCAGACTAAGAACCTAGAACCTCGCTGGAAAGGAC
CTTACACAGTCCTGCTGACCACCCCCACCGCCCTCAAAGTAGACGGCATCGCAGCTTGGATACACGCCGCCCAATGCAGCTGGG
CGAACAGCTGCTGGTGAGCAGCGTGAACCTGCCGGGCGCGCATTTTTATCCGCTGGAAAGCGCGCGCGGCGGCAGCGGCGGCAG
CGCGGGCCATCTGCCGAGCGCGGCGCCGAGCCCGCAGAAACTGGATCTGGATAAAGCGAGCAAAAAATTTAGCGGCAGCCTGAG
CTGCGAAGCGGTGAGCGGCGAACCGGCGGCGGCGAGCGCGGGCGCGCCGGCGGCGATGCTGAGCGATACCGATGCGGGCGATGC
GTTTGCGAGCGCGGCGGCGGTGGCGAAACCGGGCCCGCCGGATGGCCGCAAAGGCAGCCCGTGCGGCGAAGAAGAACTGCCGAG
CGCGGCGGCGGCGGCGGCGGCGGCGGCGGCGGCGGCGGCGGCGACCGCGCGCTATAGCATGGATAGCCTGAGCAGCGAACGCTA
TTATCTGCAGAGCCCGGGCCCGCAGGGCAGCGAACTGGCGGCGCCGTGCAGCCTGTTTCCGTATCAGGCGGCGGCGGGCGCGCC
GCATGGCCCGGTGTATCCGGCGCCGAACGGCGCGCGCTATCCGTATGGCAGCATGCTGCCGCCGGGCGGCTTTCCGGCGGCGGT
GTGCCCGCCGGGCCGCGCGCAGTTTGGCCCGGGCGCGGGCGCGGGCAGCGGCGCGGGCGGCAGCAGCGGCGGCGGCGGCGGCCC
GGGCACCTATCAGTATAGCCAGGGCGCGCCGCTGTATGGCCCGTATCCGGGCGCGGCGGCGGCGGGCAGCTGCGGCGGCCTGGG
CGGCCTGGGCGTGCCGGGCAGCGGCTTTCGCGCGCATGTGTATCTGTGCAACCGCCCGCTGTGGCTGAAATTTCATCGCCATCA
GACCGAAATGATTATTACCAAACAGGGCCGCCGCATGTTTCCGTTTCTGAGCTTTAACATTAACGGCCTGAACCCGACCGCGCA
TTATAACGTGTTTGTGGAAGTGGTGCTGGCGGATCCGAACCATTGGCGCTTTCAGGGCGGCAAATGGGTGACCTGCGGCAAAGC
GGATAACAACATGCAGGGCAACAAAATGTATGTGCATCCGGAAAGCCCGAACACCGGCAGCCATTGGATGCGCCAGGAAATTAG
CTTTGGCAAACTGAAACTGACCAACAACAAAGGCGCGAACAACAACAACACCCAGATGATTGTGCTGCAGAGCCTGCATAAATA
TCAGCCGCGCCTGCATATTGTGGAAGTGACCGAAGATGGCGTGGAAGATCTGAACGAACCGAGCAAAACCCAGACCTTTACCTT
TAGCGAAACCCAGTTTATTGCGGTGACCGCGTATCAGAACACCGATATTACCCAGCTGAAAATTGATCATAACCCGTTTGCGAA
AGGCTTTCGCGATAACTATGATAGCAGCCATCAGATTGTGCCGGGCGGCCGCTATGGCGTGCAGAGCTTTTTTCCGGAACCGTT
TGTGAACACCCTGCCGCAGGCGCGCTATTATAACGGCGAACGCACCGTGCCGCAGACCAACGGCCTGCTGAGCCCGCAGCAGAG
CGAAGAAGTGGCGAACCCGCCGCAGCGCTGGCTGGTGACCCCGGTGCAGCAGCCGGGCACCAACAAACTGGATATTAGCAGCTA
TGAAAGCGAATATACCAGCAGCACCCTGCTGCCGTATGGCATTAAAAGCCTGCCGCTGCAGACCAGCCATGCGCTGGGCTATTA
TCCGGATCCGACCTTTCCGGCGATGGCGGGCTGGGGCGGCCGCGGCAGCTATCAGCGCAAAATGGCGGCGGGCCTGCCGTGGAC
CAGCCGCACCAGCCCGACCGTGTTTAGCGAAGATCAGCTGAGCAAAGAAAAAGTGAAAGAAGAAATTGGCAGCAGCTGGATTGA
AACCCCGCCGAGCATTAAAAGCCTGGATAGCAACGATAGCGGCGTGTATACCAGCGCGTGCAAACGCCGCCGCCTGAGCCCGAG
CAACAGCAGCAACGAAAACAGCCCGAGCATTAAATGCGAAGATATTAACGCGGAAGAATATAGCAAAGATACCAGCAAAGGCAT
GGGCGGCTATTATGCGTTTTATACCACCCCGGGCAGCGGCGCGACCAACTTTAGCCTGCTGAAACAGGCGGGCGATGTGGAAGA
AAACCCGGGCCCGCCATGG.
SFG DN Eomes-P2A-h1928z:
(SEQ ID NO: 11)
AGATCTTATATGGGGCACCCCCGCCCCTTGTAAACTTCCCTGACCCTGACATGACAAGAGTTACTAACAGCCCCTCTCTCCAAG
CTCACTTACAGGCTCTCTACTTAGTCCAGCACGAAGTCTGGAGACCTCTGGCGGCAGCCTACCAAGAACAACTGGACCGACCGG
TGGTACCTCACCCTTACCGAGTCGGCGACACAGTGTGGGTCCGCCGACACCAGACTAAGAACCTAGAACCTCGCTGGAAAGGAC
CTTACACAGTCCTGCTGACCACCCCCACCGCCCTCAAAGTAGACGGCATCGCAGCTTGGATACACGCCGCCCAATGCAGCTGGG
CGAACAGCTGCTGGTGAGCAGCGTGAACCTGCCGGGCGCGCATTTTTATCCGCTGGAAAGCGCGCGCGGCGGCAGCGGCGGCAG
CGCGGGCCATCTGCCGAGCGCGGCGCCGAGCCCGCAGAAACTGGATCTGGATAAAGCGAGCAAAAAATTTAGCGGCAGCCTGAG
CTGCGAAGCGGTGAGCGGCGAACCGGCGGCGGCGAGCGCGGGCGCGCCGGCGGCGATGCTGAGCGATACCGATGCGGGCGATGC
GTTTGCGAGCGCGGCGGCGGTGGCGAAACCGGGCCCGCCGGATGGCCGCAAAGGCAGCCCGTGCGGCGAAGAAGAACTGCCGAG
CGCGGCGGCGGCGGCGGCGGCGGCGGCGGCGGCGGCGGCGGCGACCGCGCGCTATAGCATGGATAGCCTGAGCAGCGAACGCTA
TTATCTGCAGAGCCCGGGCCCGCAGGGCAGCGAACTGGCGGCGCCGTGCAGCCTGTTTCCGTATCAGGCGGCGGCGGGCGCGCC
GCATGGCCCGGTGTATCCGGCGCCGAACGGCGCGCGCTATCCGTATGGCAGCATGCTGCCGCCGGGCGGCTTTCCGGCGGCGGT
GTGCCCGCCGGGCCGCGCGCAGTTTGGCCCGGGCGCGGGCGCGGGCAGCGGCGCGGGCGGCAGCAGCGGCGGCGGCGGCGGCCC
GGGCACCTATCAGTATAGCCAGGGCGCGCCGCTGTATGGCCCGTATCCGGGCGCGGCGGCGGCGGGCAGCTGCGGCGGCCTGGG
CGGCCTGGGCGTGCCGGGCAGCGGCTTTCGCGCGCATGTGTATCTGTGCAACCGCCCGCTGTGGCTGAAATTTCATCGCCATCA
GACCGAAATGATTATTACCAAACAGGGCCGCCGCATGTTTCCGTTTCTGAGCTTTAACATTAACGGCCTGAACCCGACCGCGCA
TTATAACGTGTTTGTGGAAGTGGTGCTGGCGGATCCGAACCATTGGCGCTTTCAGGGCGGCAAATGGGTGACCTGCGGCAAAGC
GGATAACAACATGCAGGGCAACAAAATGTATGTGCATCCGGAAAGCCCGAACACCGGCAGCCATTGGATGCGCCAGGAAATTAG
CTTTGGCAAACTGAAACTGACCAACAACAAAGGCGCGAACAACAACAACACCCAGATGATTGTGCTGCAGAGCCTGCATAAATA
TCAGCCGCGCCTGCATATTGTGGAAGTGACCGAAGATGGCGTGGAAGATCTGAACGAACCGAGCAAAACCCAGACCTTTACCTT
TAGCGAAACCCAGTTTATTGCGGTGACCGCGTATCAGAACACCGATATTACCCAGCTGAAAATTGATCATAACCCGTTTGCGAA
AGGCTTTCGCGATAACTATGATAGCAGCCATCAGATTGTGCCGGGCGGCCGCTATGGCGTGCAGAGCTTTTTTCCGGAACCGTT
TGTGAACACCCTGCCGCAGGCGCGCTATTATAACGGCGAACGCACCGTGCCGCAGACCAACGGCATGGCGCTGGAAGATCGCTG
CAGCCCGCAGAGCGCGCCGAGCCCGATTACCCTGCAGATGCAGCATCTGCATCATCAGCAGCAGCAGCAGCAGCAGCAGCAGCA
GCAGATGCAGCATCTGCATCAGCTGCAGCAGCTGCAGCAGCTGCATCAGCAGCAGCTGGCGGCGGGCGTGTTTCATCATCCGGC
GATGGCGTTTGATGCGGCGGCGGCGGCGGCGGCGGCGGCGGCGGCGGCGGCGGCGCATGCGCATGCGGCGGCGCTGCAGCAGCG
CCTGAGCGGCAGCGGCAGCCCGGCGAGCTGCAGCACCCCGGCGAGCAGCACCCCGCTGACCATTAAAGAAGAAGAAAGCGATAG
CGTGATTGGCGATATGAGCTTTCATAACCAGACCCATACCACCAACGAAGAAGAAGAAGCGGAAGAAGATGATGATATTGATGT
GGATGTGGATGATACCAGCGCGGGCGGCCGCCTGCCGCCGCCGGCGCATCAGCAGCAGAGCACCGCGAAACCGAGCCTGGCGTT
TAGCATTAGCAACATTCTGAGCGATCGCTTTGGCGATGTGCAGAAACCGGGCAAAAGCATTGAAAACCAGGCGAGCATTTTTCG
CCCGTTTGAAGCGAACCGCAGCCAGACCGCGACCCCGAGCGCGTTTACCCGCGTGGATCTGCTGGAATTTAGCCGCCAGCAGCA
GGCGGCGGCGGCGGCGGCGACCGCGGCGATGATGCTGGAACGCGCGAACTTTCTGAACTGCTTTAACCCGGCGGCGTATCCGCG
CATTCATGAAGAAATTGTGCAGAGCCGCCTGCGCCGCAGCGCGGCGAACGCGGTGATTCCGCCGCCGATGAGCAGCAAAATGAG
CGATGCGAACCCGGAAAAAAGCGCGCTGGGCAGCGGCAGCGGCGCGACCAACTTTAGCCTGCTGAAACAGGCGGGCGATGTGGA
AGAAAACCCGGGCCCGCCATGG.
SFG cMyb-P2A-h1928z:
(SEQ ID NO: 12)
AGATCTTATATGGGGCACCCCCGCCCCTTGTAAACTTCCCTGACCCTGACATGACAAGAGTTACTAACAGCCCCTCTCTCCAAG
CTCACTTACAGGCTCTCTACTTAGTCCAGCACGAAGTCTGGAGACCTCTGGCGGCAGCCTACCAAGAACAACTGGACCGACCGG
TGGTACCTCACCCTTACCGAGTCGGCGACACAGTGTGGGTCCGCCGACACCAGACTAAGAACCTAGAACCTCGCTGGAAAGGAC
CTTACACAGTCCTGCTGACCACCCCCACCGCCCTCAAAGTAGACGGCATCGCAGCTTGGATACACGCCGCCCAATGGCGCGCCG
CCCGCGCCATAGCATTTATAGCAGCGATGAAGATGATGAAGATTTTGAAATGTGCGATCATGATTATGATGGCCTGCTGCCGAA
AAGCGGCAAACGCCATCTGGGCAAAACCCGCTGGACCCGCGAAGAAGATGAAAAACTGAAAAAACTGGTGGAACAGAACGGCAC
CGATGATTGGAAAGTGATTGCGAACTATCTGCCGAACCGCACCGATGTGCAGTGCCAGCATCGCTGGCAGAAAGTGCTGAACCC
GGAACTGATTAAAGGCCCGTGGACCAAAGAAGAAGATCAGCGCGTGATTGAACTGGTGCAGAAATATGGCCCGAAACGCTGGAG
CGTGATTGCGAAACATCTGAAAGGCCGCATTGGCAAACAGTGCCGCGAACGCTGGCATAACCATCTGAACCCGGAAGTGAAAAA
AACCAGCTGGACCGAAGAAGAAGATCGCATTATTTATCAGGCGCATAAACGCCTGGGCAACCGCTGGGCGGAAATTGCGAAACT
GCTGCCGGGCCGCACCGATAACGCGATTAAAAACCATTGGAACAGCACCATGCGCCGCAAAGTGGAACAGGAAGGCTATCTGCA
GGAAAGCAGCAAAGCGAGCCAGCCGGCGGTGGCGACCAGCTTTCAGAAAAACAGCCATCTGATGGGCTTTGCGCAGGCGCCGCC
GACCGCGCAGCTGCCGGCGACCGGCCAGCCGACCGTGAACAACGATTATAGCTATTATCATATTAGCGAAGCGCAGAACGTGAG
CAGCCATGTGCCGTATCCGGTGGCGCTGCATGTGAACATTGTGAACGTGCCGCAGCCGGCGGCGGCGGCGATTCAGCGCCATTA
TAACGATGAAGATCCGGAAAAAGAAAAACGCATTAAAGAACTGGAACTGCTGCTGATGAGCACCGAAAACGAACTGAAAGGCCA
GCAGGTGCTGCCGACCCAGAACCATACCTGCAGCTATCCGGGCTGGCATAGCACCACCATTGCGGATCATACCCGCCCGCATGG
CGATAGCGCGCCGGTGAGCTGCCTGGGCGAACATCATAGCACCCCGAGCCTGCCGGCGGATCCGGGCAGCCTGCCGGAAGAAAG
CGCGAGCCCGGCGCGCTGCATGATTGTGCATCAGGGCACCATTCTGGATAACGTGAAAAACCTGCTGGAATTTGCGGAAACCCT
GCAGTTTATTGATAGCTTTCTGAACACCAGCAGCAACCATGAAAACAGCGATCTGGAAATGCCGAGCCTGACCAGCACCCCGCT
GATTGGCCATAAACTGACCGTGACCACCCCGTTTCATCGCGATCAGACCGTGAAAACCCAGAAAGAAAACACCGTGTTTCGCAC
CCCGGCGATTAAACGCAGCATTCTGGAAAGCAGCCCGCGCACCCCGACCCCGTTTAAACATGCGCTGGCGGCGCAGGAAATTAA
ATATGGCCCGCTGAAAATGCTGCCGCAGACCCCGAGCCATCTGGTGGAAGATCTGCAGGATGTGATTAAACAGGAAAGCGATGA
AAGCGGCATTGTGGCGGAATTTCAGGAAAACGGCCCGCCGCTGCTGAAAAAAATTAAACAGGAAGTGGAAAGCCCGACCGATAA
AAGCGGCAACTTTTTTTGCAGCCATCATTGGGAAGGCGATAGCCTGAACACCCAGCTGTTTACCCAGACCAGCCCGGTGGCGGA
TGCGCCGAACATTCTGACCAGCAGCGTGCTGATGGCGCCGGCGAGCGAAGATGAAGATAACGTGCTGAAAGCGTTTACCGTGCC
GAAAAACCGCAGCCTGGCGAGCCCGCTGCAGCCGTGCAGCAGCACCTGGGAACCGGCGAGCTGCGGCAAAATGGAAGAACAGAT
GACCAGCAGCAGCCAGGCGCGCAAATATGTGAACGCGTTTAGCGCGCGCACCCTGGTGATGGGCAGCGGCGCGACCAACTTTAG
CCTGCTGAAACAGGCGGGCGATGTGGAAGAAAACCCGGGCCCGCCATGG.
SFG DN cMyb-P2A-h1928z:
(SEQ ID NO: 13)
AGATCTTATATGGGGCACCCCCGCCCCTTGTAAACTTCCCTGACCCTGACATGACAAGAGTTACTAACAGCCCCTCTCTCCAAG
CTCACTTACAGGCTCTCTACTTAGTCCAGCACGAAGTCTGGAGACCTCTGGCGGCAGCCTACCAAGAACAACTGGACCGACCGG
TGGTACCTCACCCTTACCGAGTCGGCGACACAGTGTGGGTCCGCCGACACCAGACTAAGAACCTAGAACCTCGCTGGAAAGGAC
CTTACACAGTCCTGCTGACCACCCCCACCGCCCTCAAAGTAGACGGCATCGCAGCTTGGATACACGCCGCCCAATGGCGCGCCG
CCCGCGCCATAGCATTTATAGCAGCGATGAAGATGATGAAGATTTTGAAATGTGCGATCATGATTATGATGGCCTGCTGCCGAA
AAGCGGCAAACGCCATCTGGGCAAAACCCGCTGGACCCGCGAAGAAGATGAAAAACTGAAAAAACTGGTGGAACAGAACGGCAC
CGATGATTGGAAAGTGATTGCGAACTATCTGCCGAACCGCACCGATGTGCAGTGCCAGCATCGCTGGCAGAAAGTGCTGAACCC
GGAACTGATTAAAGGCCCGTGGACCAAAGAAGAAGATCAGCGCGTGATTGAACTGGTGCAGAAATATGGCCCGAAACGCTGGAG
CGTGATTGCGAAACATCTGAAAGGCCGCATTGGCAAACAGTGCCGCGAACGCTGGCATAACCATCTGAACCCGGAAGTGAAAAA
AACCAGCTGGACCGAAGAAGAAGATCGCATTATTTATCAGGCGCATAAACGCCTGGGCAACCGCTGGGCGGAAATTGCGAAACT
GCTGCCGGGCCGCACCGATAACGCGATTAAAAACCATTGGAACAGCACCATGCGCCGCAAAGTGGAACAGGAAGGCTATCTGCA
GATGGCGCTGGAAGATCGCTGCAGCCCGCAGAGCGCGCCGAGCCCGATTACCCTGCAGATGCAGCATCTGCATCATCAGCAGCA
GCAGCAGCAGCAGCAGCAGCAGCAGATGCAGCATCTGCATCAGCTGCAGCAGCTGCAGCAGCTGCATCAGCAGCAGCTGGCGGC
GGGCGTGTTTCATCATCCGGCGATGGCGTTTGATGCGGCGGCGGCGGCGGCGGCGGCGGCGGCGGCGGCGGCGGCGCATGCGCA
TGCGGCGGCGCTGCAGCAGCGCCTGAGCGGCAGCGGCAGCCCGGCGAGCTGCAGCACCCCGGCGAGCAGCACCCCGCTGACCAT
TAAAGAAGAAGAAAGCGATAGCGTGATTGGCGATATGAGCTTTCATAACCAGACCCATACCACCAACGAAGAAGAAGAAGCGGA
AGAAGATGATGATATTGATGTGGATGTGGATGATACCAGCGCGGGCGGCCGCCTGCCGCCGCCGGCGCATCAGCAGCAGAGCAC
CGCGAAACCGAGCCTGGCGTTTAGCATTAGCAACATTCTGAGCGATCGCTTTGGCGATGTGCAGAAACCGGGCAAAAGCATTGA
AAACCAGGCGAGCATTTTTCGCCCGTTTGAAGCGAACCGCAGCCAGACCGCGACCCCGAGCGCGTTTACCCGCGTGGATCTGCT
GGAATTTAGCCGCCAGCAGCAGGCGGCGGCGGCGGCGGCGACCGCGGCGATGATGCTGGAACGCGCGAACTTTCTGAACTGCTT
TAACCCGGCGGCGTATCCGCGCATTCATGAAGAAATTGTGCAGAGCCGCCTGCGCCGCAGCGCGGCGAACGCGGTGATTCCGCC
GCCGATGAGCAGCAAAATGAGCGATGCGAACCCGGAAAAAAGCGCGCTGGGCAGCGGCAGCGGCGCGACCAACTTTAGCCTGCT
GAAACAGGCGGGCGATGTGGAAGAAAACCCGGGCCCGCCATGG.

### Example 3

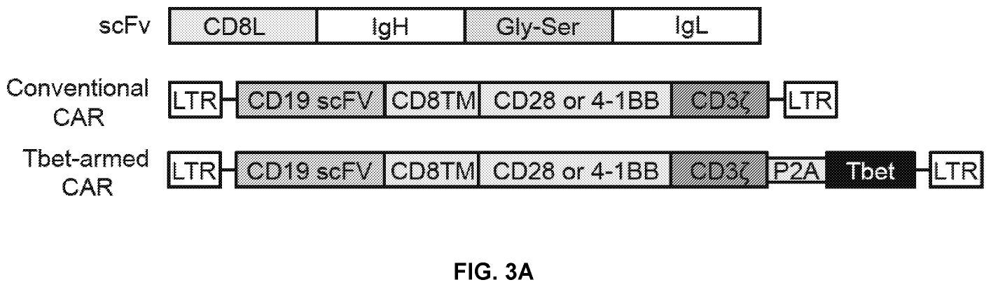

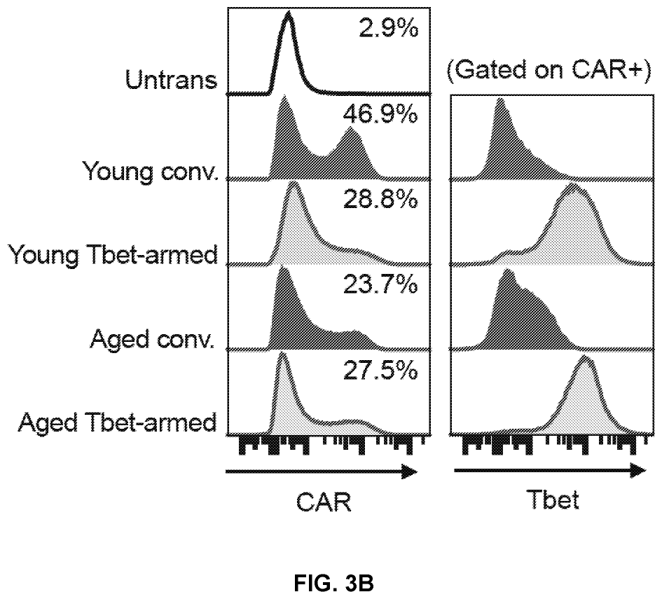

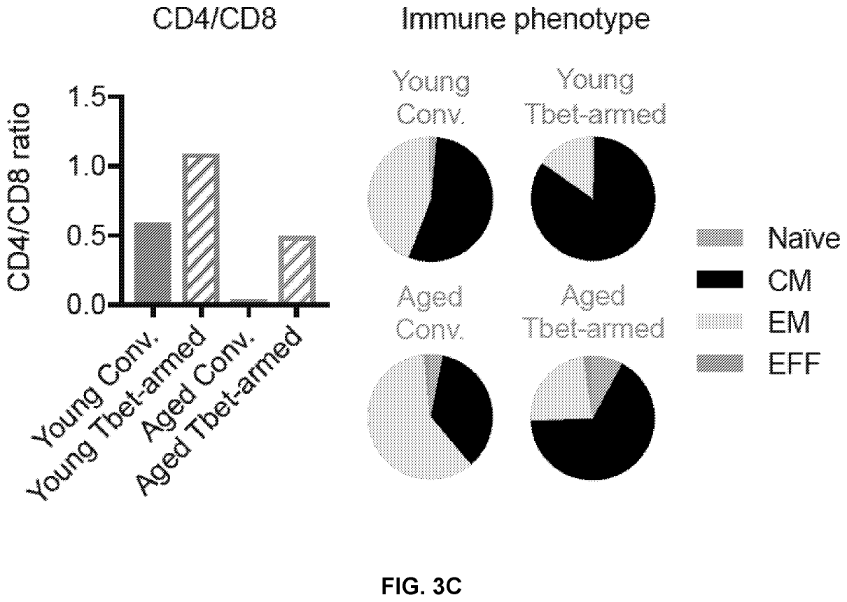

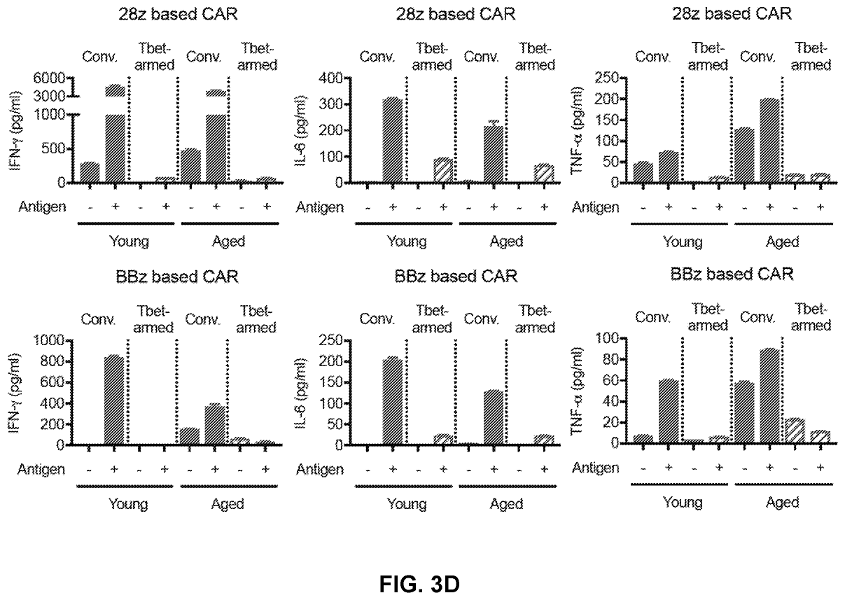

FIGS. 3A to 3D show Tbet-armed CAR exhibited unique functions via maintaining CD4 and memory-like T cells. FIG. 3A shows representation of mouse CD19-targeted CAR constructs: scFV (single-chain variable fragment), CD8L (mouse CD8 leader), IgH (immunoglobylin heavy chains), Gly-Ser (glycine-serine linker), IgL (immunoglobulin light chains), LTR (long terminal repeats), CD8TM (mouse CD8 transmembrane region), CD28 (mouse CD28 co-stimulatory domain), 4-1BB (human 4-1BB co-stimulatory domain), CD3z (CD3 ζ chain), P2A (2A self-cleaving peptides) and Tbet (mouse T-box transcription factor Tbx21). FIG. 3B shows CAR transduction efficiencies (left) and Tbet expression gated on CAR+ cells (right). Data are representative of independent experiments. FIG. 3C shows summarized immune phenotypes in each group. FIG. 3D shows cytokine production. CAR T cells were co-cultured with 3T3 (antigen-) or 3T3-mCD19 (antigen+) at 10:1 E:T ratio for 24 hours. Supernatant were collected and cytokines were measured using EIIa. Bar graphs shown as mean±% CV. For FIGS. 3B-3D shows Untrans (untransduced T cells), Young cony. (young B6 derived conventional CAR T cells), Young Tbet-armed (young B6 derived Tbet-armed CAR T cells), Aged cony. (aged B6 derived conventional CAR T cells) and Aged Tbet-armed (aged B6 derived Tbet-armed CAR T cells).

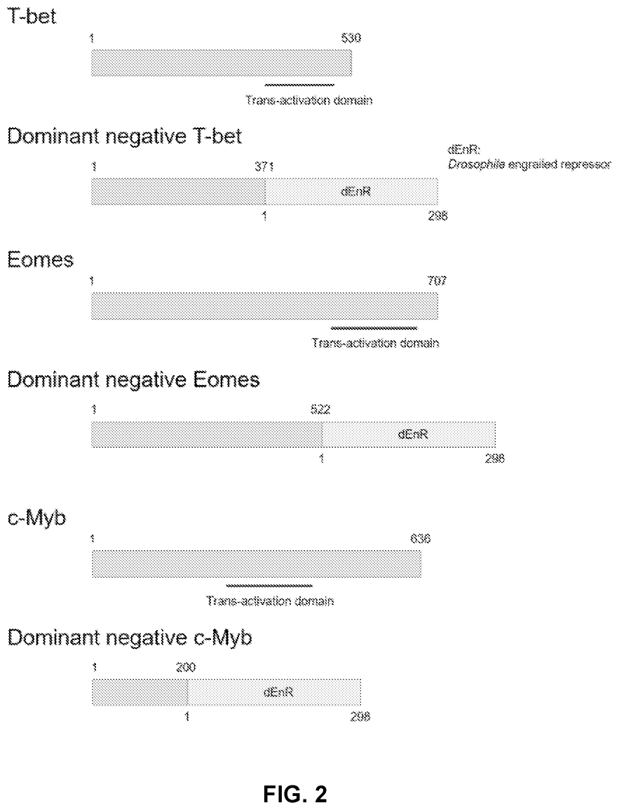

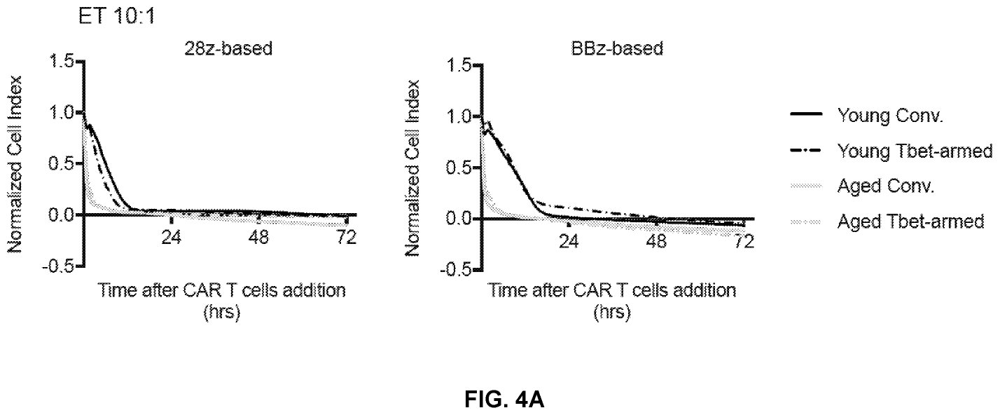

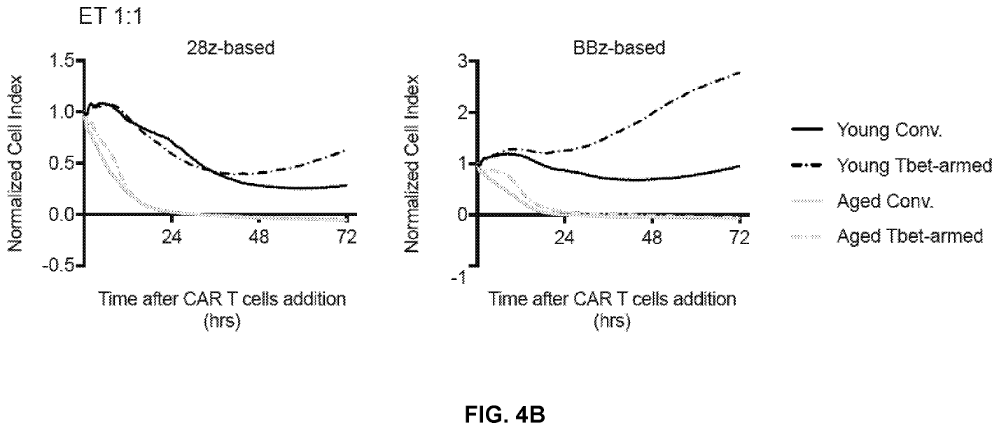

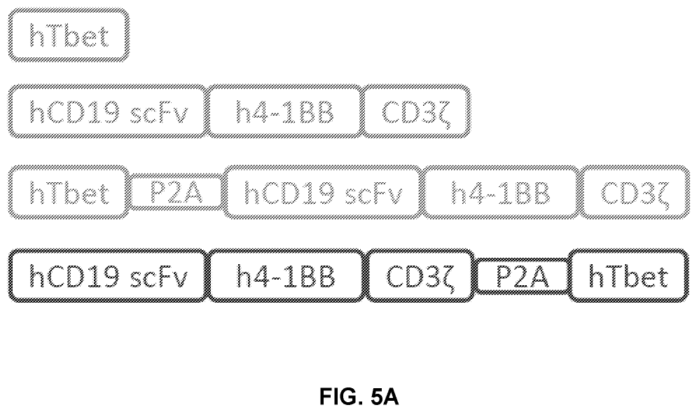

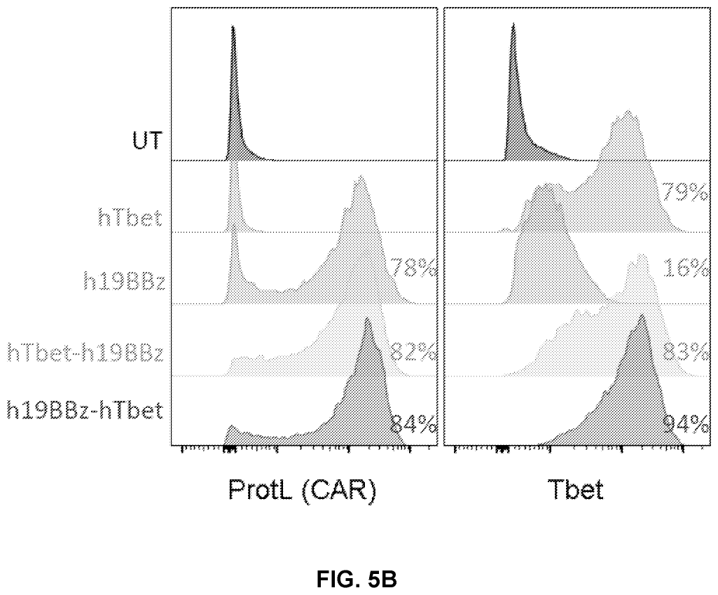

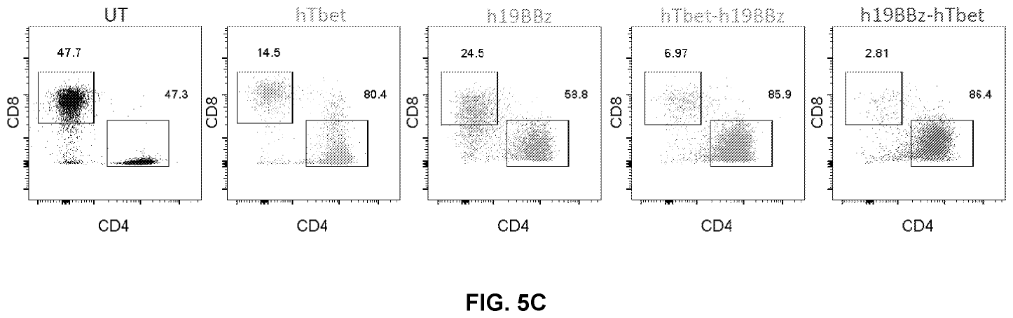

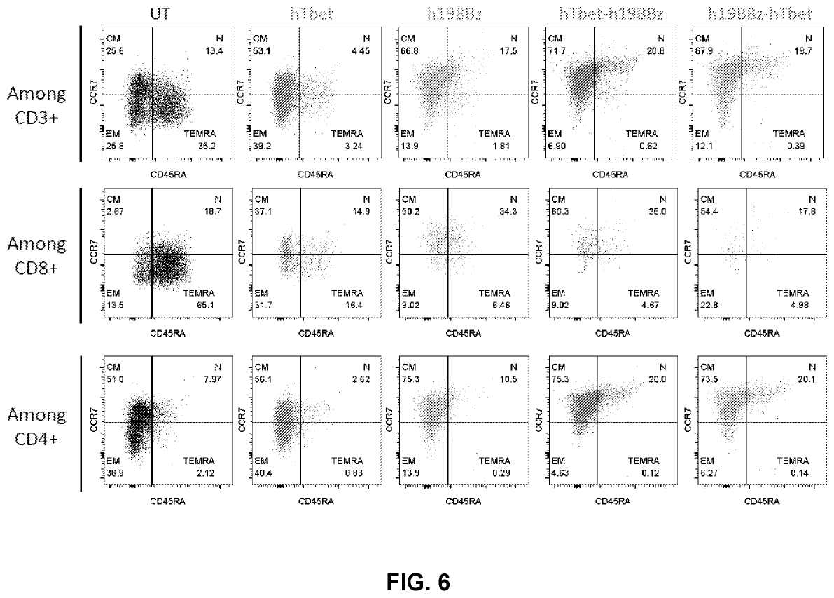

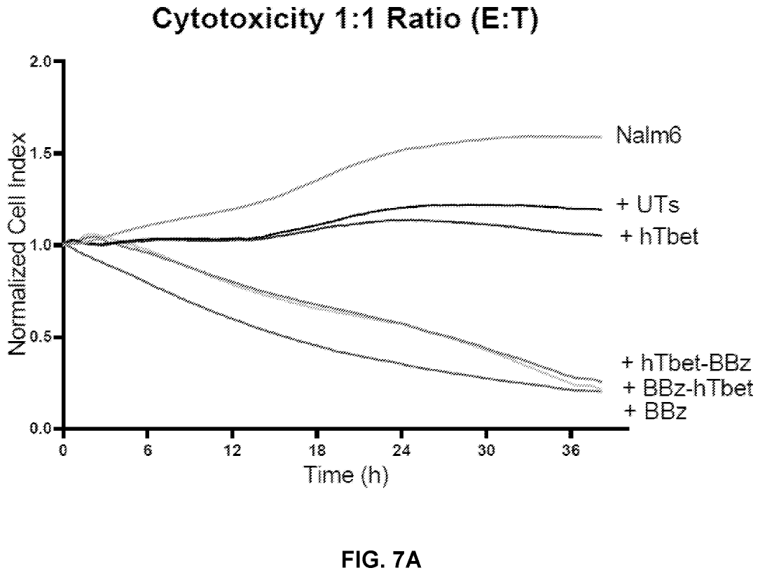

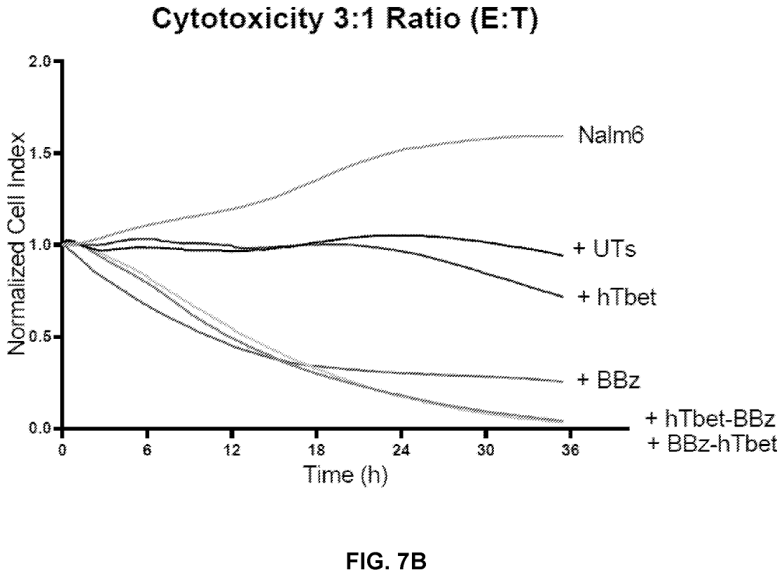

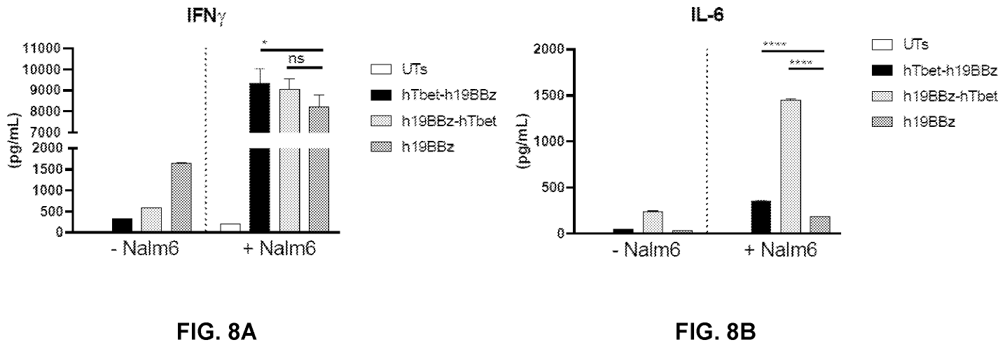

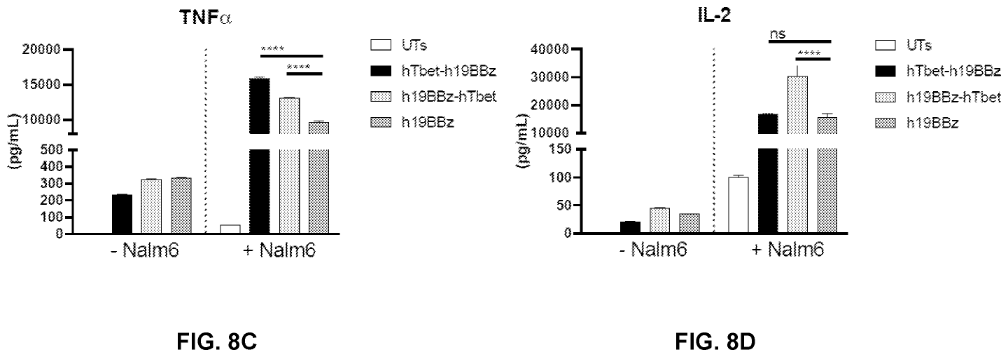

FIGS. 4A and 4B show cytotoxicity comparison of conventional mouse CAR T with Tbet-armed mouse CAR T. Cytotoxicity was monitored by xCELLigence RTCA system as well as FIG. 2D. For FIGS. 4A and 4B show CART cells co-cultured with 3T3-mCD19 at the indicated ratios. Data is representative in triplicate.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A recombinant immune effector cell genetically modified to regulate expression of one or more transcription factors selected from T-bet, Eomes, and c-Myb, wherein the recombinant immune effector cell is an aged immune effector cell, is a tumor infiltrating lymphocyte (TIL), or a combination thereof.

2. The recombinant immune effector cell of claim 1, wherein the cells are modified to express recombinant T-bet, Eomes, c-Myb, or any combination thereof.

3. The recombinant immune effector cell of claim 2, wherein the recombinant T-bet comprises the amino acid sequence SEQ ID NO:1.

4. The recombinant immune effector cell of claim 2, wherein the recombinant Eomes comprises the amino acid sequence SEQ ID NO:2.

5. The recombinant immune effector cell of claim 2, wherein the recombinant c-Myb comprises the amino acid sequence SEQ ID NO:3.

6. The recombinant immune effector cell of claim 1, wherein the cells are modified to express recombinant dominant negative forms of T-bet, Eomes, c-Myb, or any combination thereof.

7. The recombinant immune effector cell of claim 6, wherein the recombinant dominant negative T-bet comprises the amino acid sequence SEQ ID NO:4.

8. The recombinant immune effector cell of claim 2, wherein the recombinant dominant negative Eomes comprises the amino acid sequence SEQ ID NO:5.

9. The recombinant immune effector cell of claim 2, wherein the recombinant dominant negative c-Myb comprises the amino acid sequence SEQ ID NO:6.

10. The recombinant immune effector cell of claim 1, wherein the immune effector cells further expresses a chimeric antigen receptor (CAR) polypeptide.

11. The recombinant immune effector cell of claim 1, wherein the immune effector cell is selected from the group consisting of an alpha-beta T cells, a gamma-delta T cell, a Natural Killer (NK) cells, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, and a regulatory T cell.

12. The recombinant immune effector cell of claim 1, wherein the immune effector cell is a CAR-T cell.

13. The recombinant immune effector cell of claim 1, wherein the immune effector cell is a tumor infiltrating lymphocyte (TIL).

14. The recombinant immune effector cell of claim 1, wherein the aged immune effector cell is derived from a human donor at least 40 years old.

15. A method for providing an anti-tumor immunity in a subject, comprising administering to the subject an effective amount of the recombinant immune effector cell of claim 1.

16. The method of claim 15, further comprising administering to the subject a checkpoint inhibitor.

17. The method of claim 16, wherein the checkpoint inhibitor comprises an anti-PD-1 antibody, anti-PD-L1 antibody, anti-CTLA-4 antibody, or a combination thereof.

18. An ex vivo method for enhancing anti-tumor efficacy of immune effector cells, comprising genetically modifying the immune effector cells to regulate expression of one or more transcription factors selected from T-bet, Eomes, and c-Myb, in an amount effective to increase CD4/CD8 ratio by at least 0.2 when activated.

19. The ex vivo method of claim 19, wherein the immune effector cells are aged immune effector cells, tumor infiltrating lymphocyte (TIL), or a combination thereof.
